SEQUENCE LISTING (9 sequences)

SEQ ID NO: 1 - protein, 5 aa, Artificial Sequence
  from human Fab library
SYIMM

SEQ ID NO: 2 - protein, 17 aa, Artificial Sequence
  from human Fab library
SIYPSGGITFYADTVKG

SEQ ID NO: 3 - protein, 11 aa, Artificial Sequence
  from human Fab library
IKLGTVTTVDY

SEQ ID NO: 4 - protein, 14 aa, Artificial Sequence
  from human Fab library
TGTSSDVGGYNYVS

SEQ ID NO: 5 - protein, 7 aa, Artificial Sequence
  from human Fab library
DVSNRPS

SEQ ID NO: 6 - protein, 10 aa, Artificial Sequence
  from human Fab library
SSYTSSSTRV

SEQ ID NO: 7 - protein, 450 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Artificial Sequence
  from human Fab library
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 216 aa, Artificial Sequence
  from human Fab library
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2020/0048352 A1
(43) Pub. Date: Feb. 13, 2020

(54) COMBINATION OF AN ANTI-PD-L1 ANTIBODY AND A DNA-PK INHIBITOR FOR THE TREATMENT OF CANCER

(71) Applicants: Merck Patent GMBH, Darmstadt (DE); Pfizer Inc., New York, NY (US)

(72) Inventors: Astrid Zimmermann, Muehltal (DE); Lars Damstrup, Hilleroed (DE); Anne-Kathrin Prokein, Rossdorf (DE); Andreas Schroeder, Heidelberg (DE)

(21) Appl. No.: 16/498,171

(22) PCT Filed: Mar. 27, 2018
(86) PCT No.: PCT/EP2018/057708

§ 371 Date: Sep. 26, 2019

(30) Foreign Application Priority Data
Mar. 30, 2017 (EP) 17163837.2
Dec. 1, 2017 (EP) 17204926.4

(51) Int. Cl. C07K 16/28; A61K 31/5377; A61K 35/17; A61P 35/00

(52) U.S. Cl. CPC C07K 16/2827; A61K 31/5377; A61K 45/06; A61P 35/00; A61K 35/17

(57) ABSTRACT

The present invention relates to combination therapies useful for the treatment of cancer. In particular, the invention relates to a therapeutic combination which comprises an anti-PD-L antibody and a DNA-PK inhibitor, optionally together with one or more additional chemotherapeutic agents or radiotherapy. The therapeutic combination is particularly intended for use in treating a subject having a cancer that tests positive for PD-L1 expression.

### FIELD OF INVENTION

The present invention relates to combination therapies useful for the treatment of cancer. In particular, the invention relates to a therapeutic combination which comprises an anti-PD-L1 antibody and a DNA-PK inhibitor, optionally together with one or more additional chemotherapeutic agents or radiotherapy. The therapeutic combination is particularly intended for use in treating a subject having a cancer that tests positive for PD-L1 expression.

### BACKGROUND OF THE INVENTION

The mechanism of co-stimulation of T-cells has gained significant therapeutic interest in recent years for its potential to enhance cell-based immune response. Costimulatory molecules expressed on antigen-presenting cells (APCs) promote and induce T-cells to promote clonal expansion, cytokine secretion and effector function. In the absence of co-stimulation, T-cells can become refractory to antigen stimulation, do not mount an effective immune response, and further may result in exhaustion or tolerance to foreign antigens (Lenschow et al., Ann. Rev. Immunol. (1996) 14: 233). Recently, it has been discovered that T cell dysfunction or anergy occurs concurrently with an induced and sustained expression of the inhibitory receptor, programmed death-1 polypeptide (PD-1). The programmed death 1 (PD-1) receptor and PD-1 ligands 1 and 2 (PD-L1 and PD-L2, respectively) play integral roles in immune regulation. Expressed on activated T cells, PD-1 is activated by PD-L1 (also known as B7-H1) and PD-L2 expressed by stromal cells, tumor cells, or both, initiating T-cell death and localized immune suppression (Dong et al. (1999) Nat Med 5: 1365; Freeman et al. (2000) J Exp Med 192: 1027), potentially providing an immune-tolerant environment for tumor development and growth. Conversely, inhibition of this interaction can enhance local T-cell responses and mediate antitumor activity in nonclinical animal models (Iwai et al. (2002) PNAS USA 99: 12293). As a result, a number of monoclonal antibodies (mAbs) agents targeting the axis PD-1/PD-L1 are being studied for various cancers, and hundreds of clinical trials on anti-PD-1 and anti-PD-L1 mAbs are under active development.

PD-L1 is expressed in a broad range of cancers with a high frequency, up to 88% in some types of cancer. In a number of these cancers, including lung, renal, pancreatic, and ovarian cancers, the expression of PD-L1 is associated with reduced survival and an unfavorable prognosis. Interestingly, the majority of tumor infiltrating T lymphocytes predominantly express PD-1, in contrast to T lymphocytes in normal tissues and peripheral blood T lymphocytes, indicating that up-regulation of PD-1 on tumor-reactive T cells can contribute to impaired anti-tumor immune responses (Ahmadzadeh et al. (2009) Blood 14(8): 1537). This may be due to exploitation of PD-L1 signaling mediated by PD-L1 expressing tumor cells interacting with PD-1 expressing T cells to result in attenuation of T cell activation and evasion of immune surveillance (Keir et al. (2008) Annu. Rev. Immunol. 26: 677). Therefore, inhibition of the PD-L1/PD-1 interaction may enhance CD8+ T cell-mediated killing of tumors.

Similar enhancements to T cell immunity have been observed by inhibiting the binding of PD-L1 to the binding partner B7-1. Based on these finding, the blockade of PD-1/PD-L1 axis could be used therapeutically to enhance anti-tumor immune responses in patients with cancer. Hence, immune checkpoint inhibitors targeting the PD-1/PD-L1 axis have been investigated intensively in the clinical setting and have shown clinical activity in several types of cancer including melanoma, Merkel Cell Carcinoma, non-small cell lung cancer, head and neck cancer, renal cell carcinoma, urothelial carcinoma and Hodgkin's lymphoma. Although PD-1 and PD-L1 inhibitors represent significant advances in treatment and, in many cases, durable remissions, response rates have ranged between 10% and 61%, leaving many patients needing alternative therapy. Therefore, recent trends in cancer treatment are moving towards combination immunotherapy, but its success depends on addressing the challenges of finding the right drug combination, dose and schedule of the combination regimen, and managing toxicities and side effects.

DNA repair deficiency is common among tumors. Tumors with a mutational landscape dominated by C>A transversions, a pattern linked to tobacco exposure, were more likely to benefit from immune checkpoint inhibitors, and this genomic smoking signature was more predictive of immune checkpoint blockade response than patient-reported smoking history (Rizvi N A). Moreover, several of the patients who achieved durable benefit from immune checkpoint inhibitors had tumors with somatic alterations in genes involved in DNA replication or repair (such as POLE, POLD1, and MSH2).

The inhibition of PD-1 axis signaling through its direct ligands (e.g., PD-L1 or PD-L2) has been proposed as a means to enhance T cell immunity for the treatment of cancer (e.g., tumor immunity). Moreover, similar enhancements to T cell immunity have been observed by inhibiting the binding of PD-L1 to the binding partner B7-1. Furthermore, combining inhibition of PD-1 signaling with other pathways would further optimize therapeutic properties (e.g., WO 2016/205277 or WO 2016/032927).

Several clinical trials are now under way to test combinations of DNA repair-targeted agents with immune checkpoint agents in both, DNA repair-deficient and DNA repair-proficient settings. Multiple combination studies involve immune checkpoint inhibitors with DNA-damage response (DDR) inhibitors, such as poly(ADP-ribose) polymerase (PARP) and ataxia telangiectasia and RAD3-related protein (ATR) inhibitors. In addition, the success of anti-PD-1/PD-L1 therapeutics in mismatch repair-deficient tumors raises the intriguing question as to whether increasing mutational load with DDR inhibitors might increase the immunogenicity of cancers and subsequent responses to immunotherapy (Brown et al. (2017) Cancer Discovery 7(1): 20). Materials and methods for treating potentially chemoresistant tumors, e.g., using DNA-PKcs inhibitors and anti-B7-H1 antibodies are provided in WO 2016/014148.

One important class of enzymes that has been the subject of extensive study is protein kinases. Protein kinases constitute a large family of structurally related enzymes that are responsible for the control of a variety of signal transduction processes within the cell.

Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.). DNA-dependent protein kinase (DNA-PK) is a serine/threonine protein kinase which is activated in conjunction with DNA. Biochemical and genetic data show that DNA-PK consists (a) of a catalytic sub-unit, which is called DNA-PKcs, and (b) two regulatory components (Ku70 and Ku80). In functional terms, DNA-PK is a crucial constituent on the one hand of the repair of DNA double-strand breaks (DSBs) and on the other hand of somatic or V(D)J recombination. In addition, DNA-PK and its components are connected with a multiplicity of further physiological processes, including modulation of the chromatin structure and telomeric maintenance (Smith & Jackson (1999) Genes and Dev 13: 916; Goytisolo et al. (2001) Mol. Cell. Biol. 21: 3642; Williams et al. (2009) Cancer Res. 69: 2100).

Human genetic material in the form of DNA is constantly subjected to attack by reactive oxygen species (ROSs), which are formed principally as by-products of oxidative metabolism. ROSs are capable of causing DNA damage in the form of single-strand breaks. Double-strand breaks can arise if prior single-strand breaks occur in close proximity. In addition, single- and double-strand breaks may be caused if the DNA replication fork encounters damaged base patterns. Furthermore, exogenous influences, such as ionizing radiation (e.g., gamma or particle radiation), and certain anticancer medicaments (e.g., bleomycin) are capable of causing DNA double-strand breaks. DSBs may furthermore occur as intermediates of somatic recombination, a process which is important for the formation of a functional immune system of all vertebrates. If DNA double-strand breaks are not repaired or are repaired incorrectly, mutations and/or chromosome aberrations may occur, which may consequently result in cell death. In order to counter the severe dangers resulting from DNA double-strand breaks, eukaryotic cells have developed a number of mechanisms to repair them. Higher eukaryotes use predominantly so-called non-homologous end-joining, in which the DNA-dependent protein kinase adopts the key role. Biochemical investigations have shown that DNA-PK is activated most effectively by the occurrence of DNA-DSBs. Cell lines whose DNA-PK components have mutated and are non-functional prove to be radiation-sensitive.

Many diseases are associated with abnormal cellular responses, proliferation and evasion of programmed cell-death, triggered by mediated events as described above and herein. Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. If a cancer spreads (metastasizes), the new tumor bears the same name as the original (primary) tumor. The frequency of a particular cancer may depend on the gender.

Accordingly, there remains a need to develop novel therapeutic options for the treatment of cancers. Furthermore, there is a need for therapies having greater efficacy than existing therapies. Preferred combination therapies of the present invention show greater efficacy than treatment with either therapeutic agent alone.

### SUMMARY OF THE INVENTION

The present invention arises out of the discovery that a subject having a cancer can be treated with a combination comprising an anti-PD-L1 antibody and a DNA-PK inhibitor. Thus, in a first aspect, the present invention provides a method comprising administering to the subject an anti-PD-L1 antibody and a DNA-PK inhibitor for treating a cancer in a subject in need thereof. Also provided are methods of inhibiting tumor growth or progression in a subject who has malignant tumors. Also provided are methods of inhibiting metastasis of malignant cells in a subject. Also provided are methods of decreasing the risk of metastasis development and/or metastasis growth in a subject. Also provided are methods of inducing tumor regression in a subject who has malignant cells. The combination treatment results in an objective response, preferably a complete response or partial response in the subject. In some embodiments, the cancer is identified as PD-L1 positive cancerous disease.

Specific types of cancer to be treated according to the invention include, but are not limited to, cancer of the lung, head and neck, colon, neuroendocrine system, mesenchyme, breast, pancreatic, and histological subtypes thereof. In some embodiments, the cancer is selected from small-cell lung cancer (SCLC), non-small-cell lung cancer (NSCLC), squamous cell carcinoma of the head and neck (SCCHN), colorectal cancer (CRC), primary neuroendocrine tumors and sarcoma.

The anti-PD-L1 antibody and DNA-PK inhibitor can be administered in a first-line, second-line or higher treatment (i.e., beyond therapy in subjects) of the cancer. In some embodiments, SCLC extensive disease (ED), NSCLC and SCCHN are selected for first-line treatment. In some embodiments, the cancer is resistant or became resistant to prior cancer therapy. The combination therapy of the invention can also be used in the treatment of a subject with the cancer who has been previously treated with one or more chemotherapies or underwent radiotherapy but failed with such previous treatment. The cancer for second-line or beyond treatment can be pre-treated relapsing metastatic NSCLC, unresectable locally advanced NSCLC, SCLC ED, pre-treated SCLC ED, SCLC unsuitable for systemic treatment, pre-treated relapsing or metastatic SCCHN, recurrent SCCHN eligible for re-irradiation, pre-treated microsatellite status instable low (MSI-L) or microsatellite status stable (MSS) metastatic colorectal cancer (mCRC), pre-treated subset of patients with mCRC (i.e., MSI-L or MSS), and unresectable or metastatic microsatellite instable high (MSI-H) or mismatch repair-deficient solid tumors progressing after prior treatment and which have no satisfactory alternative treatment options. In some embodiments, advanced or metastatic MSI-H or mismatch repair-deficient solid tumors progressing after prior treatment and which have no satisfactory alternative treatment options, are treated with the combination of anti-PD-L1 antibody and DNA-PK inhibitor.

In some embodiments, the anti-PD-L1 antibody is used in the treatment of a human subject. In some embodiments, PD-L1 is human PD-L1.

In some embodiments, the anti-PD-L1 antibody comprises a heavy chain, which comprises three complementarity determining regions (CDRs) having amino acid sequences of SEQ ID NOs: 1, 2 and 3, and a light chain, which comprises three complementarity determining regions (CDRs) having amino acid sequences of SEQ ID NOs: 4, 5 and 6. The anti-PD-L1 antibody preferably comprises the heavy chain having amino acid sequences of SEQ ID NOs: 7 or 8 and the light chain having amino acid sequence of SEQ ID NO: 9. In some preferred embodiments, the anti-PD-L1 antibody is avelumab.

In some embodiment, the anti-PD-L1 antibody is administered intravenously (e.g., as an intravenous infusion) or subcutaneously, preferably intravenously. More preferably, the anti-PD-L1 antibody is administered as an intravenous infusion. Most preferably, the inhibitor is administered for 50-80 minutes, highly preferably as a one-hour intravenous infusion. In some embodiment, the anti-PD-L1 antibody is administered at a dose of about 10 mg/kg body weight every other week (i.e., every two weeks, or “Q2W”). In some embodiments, the anti-PD-L1 antibody is administered at a fixed dosing regimen of 800 mg as a 1 hour IV infusion Q2W.

In some aspects, the DNA-PK inhibitor is (S)-[2-chloro-4-fluoro-5-(7-morpholin-4-yl-quinazolin-4-yl)-phenyl]-(6-methoxypyridazin-3-yl)-methanol (“Compound 1”) or a pharmaceutically acceptable salt thereof. In some embodiments, the DNA-PK inhibitor is administered orally. In some embodiments, the DNA-PK inhibitor is administered at a dose of about 1 to 800 mg once or twice daily (i.e., “QD” or “BID”). Preferably, the DNA-PK inhibitor is administered at a dose of about 100 mg QD, 200 mg QD, 150 mg BID, 200 mg BID, 300 mg BID or 400 mg BID, more preferably about 400 mg BID.

In a preferred embodiment, the recommended phase II dose for the DNA-PK inhibitor is 400 mg orally twice daily, and the recommended phase II dose for avelumab is 10 mg/kg IV every second week. In a preferred embodiment, the recommended phase II dose for the DNA-PK inhibitor is 400 mg twice daily as capsule, and the recommended phase II dose for avelumab is 800 mg Q2W.

In other embodiments, the anti-PD-L1 antibody and DNA-PK inhibitor are used in combination with chemotherapy (CT), radiotherapy (RT) or chemoradiotherapy (CRT). The chemotherapeutic agent can be etoposide, doxorubicin, topotecan, irinotecan, fluorouracil, a platin, an anthracycline, and a combination thereof. In a preferred embodiment, the chemotherapeutic agent can be doxorubicin. Preclinical studies showed an anti-tumor synergistic effect with DNA-PK inhibitors without adding a major toxicity.

In some embodiments, the etoposide is administered via intravenous infusion over about 1 hour. In some embodiments, the etoposide is administered on day 1 to 3 every three weeks (i.e., “D1-3Q3W”) in an amount of about 100 mg/m². In some embodiments, the cisplatin is administered via intravenous infusion over about 1 hour. In some embodiments, the cisplatin is administered once every three weeks (i.e., “Q3W”) in an amount of about at 75 mg/m². In some embodiments, both etoposide and cisplatin are administered sequentially (at separate times) in either order or substantially simultaneously (at the same time).

In some embodiments, doxorubicin is administered every 21-28 days in an amount of 40 to 60 mg/m² IV. The dose and administration schedule could vary depending on the kind of tumor and the existing diseases and marrow reserves.

In some embodiments, the topotecan is administered on day 1 to 5 every three weeks (i.e., “D1-5Q3W”).

In some embodiments, the anthracycline is administered until reaching a maximal life-long accumulative dose.

The radiotherapy can be a treatment given with electrons, photons, protons, alfa-emitters, other ions, radio-nucleotides, boron capture neutrons and combinations thereof. In some embodiments, the radiotherapy comprises about 35-70 Gy/20-35 fractions.

In a further aspect, the combination regimen comprises a lead phase, optionally followed by a maintenance phase (or consolidation phase) after completion of the lead phase. The treatment regimens can differ in both phases. In some embodiments, the treatment regimens differ in both phases. In some embodiments, the anti-PD-L1 antibody and the DNA-PK inhibitor are administered concurrently (during the same phase) in either the lead or maintenance phase. In some embodiments, either the anti-PD-L1 antibody or the DNA-PK inhibitor can be additionally administered in the other phase, optionally together with chemotherapy, radiotherapy or chemoradiotherapy. In some embodiments, the anti-PD-L1 antibody and DNA-PK inhibitor are administered non-concurrently in the lead and maintenance phase. The concurrent administration comprises the administration of the anti-PD-L1 antibody and DNA-PK inhibitor sequentially in either order (i.e., one treatment is given after the other) or substantially simultaneously (i.e., both treatment are substantially given at the same time) in the very same phase of treatment. The non-concurrent administration comprises the administration of the anti-PD-L1 antibody and DNA-PK inhibitor sequentially in two different phases of treatment.

In some embodiments, the DNA-PK inhibitor is administered alone in the lead phase. In some embodiments, the DNA-PK inhibitor is administered concurrently with one or more therapies in the lead phase. Such therapies can involve an anti-PD-L1 antibody, a chemotherapy or radiotherapy, or a combination thereof. The lead phase particularly comprises the concurrent administration of the DNA-PK inhibitor and PD-L1 antibody.

In some embodiments, there is no maintenance phase. In some embodiments, neither the anti-PD-L1 antibody nor the DNA-PK inhibitor is administered in the maintenance phase. In some embodiments, the anti-PD-L1 antibody is administered alone in the maintenance phase. In some embodiments, the anti-PD-L1 antibody is administered concurrently with the DNA-PK inhibitor in the maintenance phase.

In some embodiments, the lead phase comprises the administration of the DNA-PK inhibitor and, after completion of the lead phase, the maintenance phase comprises the administration of the anti-PD-L1 antibody. Both, the DNA-PK inhibitor and anti-PD-L1 antibody can be administered alone or concurrently with one or more chemotherapeutic agents, radiotherapy or chemoradiotherapy.

In some preferred embodiments, SCLC ED is treated in the lead phase comprising the concurrent administration of the DNA-PK inhibitor and etoposide, optionally together with cisplatin, and, after completion of the lead phase, in the maintenance phase comprising the administration of the anti-PD-L1 antibody, optionally together with the DNA-PK inhibitor. Herein, the lead phase particularly comprises the triple combination of the DNA-PK inhibitor, etoposide and cisplatin for SCLC ED treatment. In some other preferred embodiments, SCLC ED is treated in the lead phase comprising the concurrent administration of the anti-PD-L1 antibody, DNA-PK inhibitor and etoposide, optionally together with cisplatin. Herein, the lead phase particularly comprises the quadruple combination of the anti-PD-L1 antibody, DNA-PK inhibitor, etoposide and cisplatin for SCLC ED treatment. After completion of the lead phase, the SCLC ED treatment can be continued in the maintenance phase comprising the administration of the anti-PD-L1 antibody. In some embodiments, the etoposide, optionally together with cisplatin, is administered up to 6 cycles or until progression of SCLC ED.

In some other preferred embodiments, mCRC MSI-L is treated in the lead phase comprising the concurrent administration of the anti-PD-L1 antibody, DNA-PK inhibitor, irinotecan and fluorouracil.

In some other preferred embodiments, NSCLC or SCCHN is treated in the lead phase comprising the concurrent administration of the DNA-PK inhibitor and radiotherapy or chemoradiotherapy and, after completion of the lead phase, in the maintenance phase comprising the administration of the anti-PD-L1 antibody. Herein, the lead phase particularly comprises the concurrent administration of the anti-PD-L1 antibody, DNA-PK inhibitor and radiotherapy for the NSCLC or SCCHN treatment.

In a further aspect, the invention also relates to a method for advertising an anti-PD-L1 antibody in combination with a DNA-PK inhibitor, comprising promoting, to a target audience, the use of the combination for treating a subject with a cancer based on PD-L1 expression in samples, preferably tumor samples, taken from the subject. The PD-L1 expression can be determined by immunohistochemistry, e.g., using one or more primary anti-PD-L1 antibodies.

Provided herein is also a pharmaceutical composition comprising an anti-PD-L1 antibody, a DNA-PK inhibitor and at least a pharmaceutically acceptable excipient or adjuvant. The anti-PD-L1 antibody and the DNA-PK inhibitor are provided in a single or separate unit dosage forms.

Also provided herein is an anti-PD-L1 antibody in combination with a DNA-PK inhibitor for use as a medicament, particularly for use in the treatment of cancer. Similarly, a DNA-PK inhibitor is provided in combination with an anti-PD-L1 antibody for use as a medicament, particularly for use in the treatment of cancer. Also provided is a combination comprising an anti-PD-L1 antibody and a DNA-PK inhibitor for any purpose, for use as a medicament or in the treatment of cancer. Also provided is the use of a combination for the manufacture of a medicament for the treatment of cancer, comprising an anti-PD-L1 antibody and a DNA-PK inhibitor.

In a further aspect, the invention relates to a kit comprising an anti-PD-L1 antibody and a package insert comprising instructions for using the anti-PD-L1 antibody in combination with a DNA-PK inhibitor to treat or delay progression of a cancer in a subject. Also provided is a kit comprising a DNA-PK inhibitor and a package insert comprising instructions for using the DNA-PK inhibitor in combination with an anti-PD-L1 antibody to treat or delay progression of a cancer in a subject. Also provided is a kit comprising an anti-PD-L1 antibody and a DNA-PK inhibitor, and a package insert comprising instructions for using the anti-PD-L1 antibody and a DNA-PK inhibitor to treat or delay progression of a cancer in a subject. The kit can comprise a first container, a second container and a package insert, wherein the first container comprises at least one dose of a medicament comprising an anti-PD-L1 antibody, the second container comprises at least one dose of a medicament comprising a DNA-PK inhibitor, and the package insert comprises instructions for treating a subject for cancer using the medicaments. The instructions can state that the medicaments are intended for use in treating a subject having a cancer that tests positive for PD-L1 expression by an immunohistochemical (IHC) assay.

In various embodiments, the anti-PD-L1 antibody administered to the subject is avelumab and/or the DNA-PK inhibitor is (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof.

### BRIEF DESCRIPTION OF THE FIGURES

FIG. 1 shows the heavy chain sequence of avelumab. (A) SEQ ID NO: 7 represents the full length heavy chain sequence of avelumab. The CDRs having the amino acid sequences of SEQ ID NOs: 1, 2 and 3 are marked by underlining. (B) SEQ ID NO: 8 represents the heavy chain sequence of avelumab without the C-terminal lysine. The CDRs having the amino acid sequences of SEQ ID NOs: 1, 2 and 3 are marked by underlining.

FIG. 2 (SEQ ID NO: 9) shows the light chain sequence of avelumab. The CDRs having the amino acid sequences of SEQ ID NOs: 4, 5 and 6 are marked by underlining.

FIG. 3 shows that Compound 1 (aka M3814) in combination with avelumab (without DNA damaging agent) increased the tumor growth inhibition and improved survival compared to single agent treatments in a syngeneic MC38 tumor model. M3814 was applied daily started from day 0; Avelumab was applied on days 3, 6 and 9.

FIG. 4 shows that a combination of radiotherapy, M3814 and avelumab resulted in a superior tumor growth control versus radiotherapy alone, radiotherapy and M3814, or radiotherapy and avelumab, in the syngeneic MC38 model.

FIG. 5 shows options to include avelumab in 1 L SCLC development. (1) Additional 3^{rd} arm in MS100036-0022 with CT+M3814+(maintenance avelumab, or maintenance avelumab+M3814) for patients who receive clinical benefit (SD, PR or CR); (2) 4-arm trial (concurrent) with CT+/−M3814+/−avelumab (factorial design, which allows evaluation of contribution of each drug to the combination effect); (3) separate trial (CT+avelumab+/−M3814) and plan for pooled analyses. A multicenter trial with an open label phase Ib part is followed by a randomized, placebo-controlled, double-blind, phase II part to evaluate efficacy, safety, tolerability, and PK of the DNA-PK inhibitor M3814 and avelumab in combination with etoposide and cisplatin in subjects with SCLC ED.

FIG. 6 shows an option to include avelumab in 1 L SCLC development with a combination of CT+M3814+avelumab concurrent as 3^{1d} arm.

FIG. 7 shows an option to include avelumab in 1 L SCLC development with a quadruple combination followed by avelumab maintenance (all arms).

FIG. 8 shows a development opportunity for avelumab+M3814 with CT: Potential phase II trial in 2 L SCLC ED.

FIG. 9 shows a development opportunity for avelumab+M3814 without RT: Combination with SoC in patients with mCRC MSI low.

FIG. 10 shows a phase 1 b dose escalation study: Avelumab+M3814 (DNA-PKi). (1) Indication expansion: 2 L CRC MSI low; (2) Indication expansion: 1 L/2 L SCCHN and 1 L/2 L NSCLC.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

“A”, “an”, and “the” include plural referents unless the context clearly dictates otherwise. Thus, for example, reference to an antibody refers to one or more antibodies or at least one antibody. As such, the terms “a” (or “an”), “one or more”, and “at least one” are used interchangeably herein.

“About” when used to modify a numerically defined parameter (e.g., the dose of an anti-PD-L1 antibody or DNA-PK inhibitor, or the length of treatment time with a combination therapy described herein) means that the parameter may vary by as much as 10% below or above the stated numerical value for that parameter. For example, a dose of about 10 mg/kg may vary between 9 mg/kg and 11 mg/kg.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies).

“Antigen-binding fragment” of an antibody or “antibody fragment” comprises a portion of an intact antibody, which is still capable of antigen binding and/or the variable region of the intact antibody. Antigen-binding fragments include, for example, Fab, Fab′, F(ab′)2, Fd, and Fv fragments, domain antibodies (dAbs, e.g., shark and camelid antibodies), fragments including complementarity determining regions (CDRs), single chain variable fragment antibodies (scFv), single-chain antibody molecules, multi-specific antibodies formed from antibody fragments, maxibodies, minibodies, intrabodies, diabodies, triabodies, tetrabodies, v-NAR and bis-scFv, linear antibodies (see e.g., U.S. Pat. No. 5,641,870, Example 2; Zapata et al. (1995) Protein Eng. 8H0: 1057), and polypeptides that contain at least a portion of an immunoglobulin that is sufficient to confer specific antigen binding to the polypeptide. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment, which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments were originally produced as pairs of Fab′ fragments which have hinge cysteines between them. Other chemical couplings of antibody fragments are also known.

“Antibody-dependent cell-mediated cytotoxicity” or “ADCC” refers to a form of cytotoxicity in which secreted Ig bound onto Fc receptors (FcRs) present on certain cytotoxic cells (e.g., natural killer (NK) cells, neutrophils, and macrophages) enable these cytotoxic effector cells to bind specifically to an antigen-bearing target cell and subsequently kill the target cell with cytotoxins. The antibodies arm the cytotoxic cells and are required for killing of the target cell by this mechanism. The primary cells for mediating ADCC, the NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991).

“Anti-PD-L1 antibody” means an antibody that blocks binding of PD-L1 expressed on a cancer cell to PD-1. In any of the treatment method, medicaments and uses of the present invention in which a human subject is being treated, the anti-PD-L1 antibody specifically binds to human PD-L1 and blocks binding of human PD-L1 to human PD-1. The antibody may be a monoclonal antibody, human antibody, humanized antibody or chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen-binding fragment is selected from the group consisting of Fab, Fab′-SH, F(ab′)₂, scFv and Fv fragments. Examples of monoclonal antibodies that bind to human PD-L1, and useful in the treatment method, medicaments and uses of the present invention, are described in WO 2007/005874, WO 2010/036959, WO 2010/077634, WO 2010/089411, WO 2013/019906, WO 2013/079174, WO 2014/100079, WO 2015/061668, and U.S. Pat. Nos. 8,552,154, 8,779,108 and 8,383,796. Specific anti-human PD-L1 monoclonal antibodies useful as the PD-L1 antibody in the treatment method, medicaments and uses of the present invention include, for example without limitation, avelumab (MSB0010718C), nivolumab (BMS-936558), MPDL3280A (an IgG1-engineered, anti-PD-L1 antibody), BMS-936559 (a fully human, anti-PD-L1, IgG4 monoclonal antibody), MED14736 (an engineered IgG1 kappa monoclonal antibody with triple mutations in the Fc domain to remove antibody-dependent, cell-mediated cytotoxic activity), and an antibody which comprises the heavy chain and light chain variable regions of SEQ ID NO:24 and SEQ ID NO:21, respectively, of WO 2013/019906.

“Biomarker” generally refers to biological molecules, and quantitative and qualitative measurements of the same, that are indicative of a disease state. “Prognostic biomarkers” correlate with disease outcome, independent of therapy. For example, tumor hypoxia is a negative prognostic marker—the higher the tumor hypoxia, the higher the likelihood that the outcome of the disease will be negative. “Predictive biomarkers” indicate whether a patient is likely to respond positively to a particular therapy. E.g., HER2 profiling is commonly used in breast cancer patients to determine if those patients are likely to respond to Herceptin (trastuzumab, Genentech). “Response biomarkers” provide a measure of the response to a therapy and so provide an indication of whether a therapy is working. For example, decreasing levels of prostate-specific antigen generally indicate that anti-cancer therapy for a prostate cancer patient is working. When a marker is used as a basis for identifying or selecting a patient for a treatment described herein, the marker can be measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of a biomarker in a clinical setting is a clear indication that this parameter was used as a basis for initiating, continuing, adjusting and/or ceasing administration of the treatments described herein.

“Blood” refers to all components of blood circulating in a subject including, but not limited to, red blood cells, white blood cells, plasma, clotting factors, small proteins, platelets and/or cryoprecipitate. This is typically the type of blood which is donated when a human patient gives blood. Plasma is known in the art as the yellow liquid component of blood, in which the blood cells in whole blood are typically suspended. It makes up about 55% of the total blood volume. Blood plasma can be prepared by spinning a tube of fresh blood containing an anti-coagulant in a centrifuge until the blood cells fall to the bottom of the tube. The blood plasma is then poured or drawn off. Blood plasma has a density of approximately 1025 kg/m³ or 1.025 kg/I.

“Cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia, multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

“Chemotherapy” is a therapy involving a chemotherapeutic agent, which is a chemical compound useful in the treatment of cancer. Examples of chemotherapeutic agents include alkylating agents such as thiotepa and cyclophosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethiylenethiophosphoramide, and trimethylolomelamine; acetogenins (especially bullatacin and bullatacinone); delta-9-tetrahydrocannabinol (dronabinol); beta-lapachone; lapachol; colchicines; betulinic acid; a camptothecin (including the synthetic analogue topotecan (CPT-11 (irinotecan), acetylcamptothecin, scopolectin, and 9-aminocamptothecin); bryostatin; pemetrexed; callystatin; CC-1065 (including its adozelesin, carzelesin, and bizelesin synthetic analogues); podophyllotoxin; podophyllinic acid; teniposide; cryptophycins (particularly, cryptophycin 1 and cryptophycin 8); dolastatin; duocarmycin (including the synthetic analogues KW-2189 and CB1-TM1); eleutherobin; pancratistatin; TLK-286; CDP323, an oral alpha-4 integrin inhibitor; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Nicolaou et al. (1994) Angew. Chem Intl. Ed. Engl. 33: 183); dynemicin including dynemicin A; an esperamicin; as well as neocarzinostatin chromophore and related chromoprotein enediyne antibiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, doxorubicin HCl liposome injection, and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites such as methotrexate, gemcitabine, tegafur, capecitabine, an epothilone, and 5-fluorouracil (5-FU); folic acid analogues such as denopterin, methotrexate, pteropterin, and trimetrexate; purine analogs such as fludarabine, 6-mercaptopurine, thiamiprine, and thioguanine; pyrimidine analogs such as ancitabine, azacitidine, 6-azauridine, carmofur, cytarabine, dideoxyuridine, doxifluridine, enocitabine, floxuridine, and imatinib (a 2-phenylaminopyrimidine derivative), as well as other c-Kit inhibitors; anti-adrenals such as aminoglutethimide, mitotane, and trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially, T-2 toxin, verracurin A, roridin A, and anguidine); urethan; vindesine; dacarbazine; mannomustine; mitobronitol; mitolactol; pipobroman; gacytosine; arabinoside (“Ara-C”); thiotepa; taxoids, e.g., paclitaxel, albumin-engineered nanoparticle formulation of paclitaxel, and doxetaxel; chloranbucil; 6-thioguanine; mercaptopurine; methotrexate; platinum analogs such as cisplatin and carboplatin; vinblastine; platinum; etoposide (VP-16); ifosfamide; mitoxantrone; vincristine; oxaliplatin; leucovovin; vinorelbine; novantrone; edatrexate; daunomycin; aminopterin; ibandronate; topoisomerase inhibitor RFS 2000; difluoromethylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine and prednisolone, or FOLFOX, an abbreviation for a treatment regimen with oxaliplatin combined with 5-FU and leucovovin.

“Clinical outcome”, “clinical parameter”, “clinical response”, or “clinical endpoint” refers to any clinical observation or measurement relating to a patient's reaction to a therapy. Non-limiting examples of clinical outcomes include tumor response (TR), overall survival (OS), progression free survival (PFS), disease free survival, time to tumor recurrence (TTR), time to tumor progression (TTP), relative risk (RR), toxicity, or side effect.

“Complete response” or “complete remission” refers to the disappearance of all signs of cancer in response to treatment. This does not always mean the cancer has been cured.

“Comprising”, as used herein, is intended to mean that the compositions and methods include the recited elements, but not excluding others. “Consisting essentially of”, when used to define compositions and methods, shall mean excluding other elements of any essential significance to the composition or method. “Consisting of” shall mean excluding more than trace elements of other ingredients for claimed compositions and substantial method steps.

Embodiments defined by each of these transition terms are within the scope of this invention. Accordingly, it is intended that the methods and compositions can include additional steps and components (comprising) or alternatively including steps and compositions of no significance (consisting essentially of) or alternatively, intending only the stated method steps or compositions (consisting of).

“Dose” and “dosage” refer to a specific amount of active or therapeutic agents for administration. Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

“Diabodies” refer to small antibody fragments prepared by constructing sFv fragments with short linkers (about 5-10 residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments, in which the V_{H} and V_{L} domains of the two antibodies are present on different polypeptide chains. Diabodies are described in greater detail in, for example, EP 404097; WO 1993/11161; Hollinger et al. (1993) PNAS USA 90: 6444.

“Enhancing T-cell function” means to induce, cause or stimulate a T-cell to have a sustained or amplified biological function, or renew or reactivate exhausted or inactive T-cells. Examples of enhancing T-cell function include: increased secretion of y-interferon from CD8+ T-cells, increased proliferation, increased antigen responsiveness (e.g., viral, pathogen, or tumor clearance) relative to such levels before the intervention. In one embodiment, the level of. enhancement is as least 50%, alternatively 60%, 70%, 80%, 90%, 100%, 120%, 150%, 200%. The manner of measuring this enhancement is known to one of ordinary skill in the art.

“Fc” is a fragment comprising the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen-binding or variable region of the intact antibody or the Fc region of an antibody which retains or has modified FcR binding capability. Examples of functional antibody fragments include linear antibodies, single-chain antibody molecules, and multi-specific antibodies formed from antibody fragments.

“Fv” is the minimum antibody fragment, which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen-binding specificity to the antibody. However, even a single variable domain (or half of an Fv comprising only three HVRs specific for an antigen) has the ability to recognize and bind antigen, although at a lower affinity than the entire binding site.

“Human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries (see e.g., Hoogenboom and Winter (1991), JMB 227: 381; Marks et al. (1991) JMB 222: 581). Also available for the preparation of human monoclonal antibodies are methods described in Cole et al. (1985) Monoclonal Antibodies and Cancer Therapy, Alan R. Liss, page 77; Boerner et al. (1991), J. Immunol 147(1): 86; van Dijk and van de Winkel (2001) Curr. Opin. Pharmacol 5: 368). Human antibodies can be prepared by administering the antigen to a transgenic animal that has been modified to produce such antibodies in response to antigenic challenge but whose endogenous loci have been disabled, e.g., immunized xenomice (see e.g., U.S. Pat. Nos. 6,075,181; and 6,150,584 regarding XENOMOUSE technology). See also, for example, Li et al. (2006) PNAS USA, 103: 3557, regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR of the recipient are replaced by residues from an HVR of a non-human species (donor antibody) such as mouse, rat, rabbit, or non-human primate having the desired specificity, affinity and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and no more than 3 in the L chain. The humanized antibody optionally will also comprise at least a portion of an immunoglobulin constant region (Fc), typically that of a human immunoglobulin. For further details, see e.g., Jones et al. (1986) Nature 321: 522; Riechmann et al. (1988), Nature 332: 323; Presta (1992) Curr. Op. Struct. Biol. 2: 593; Vaswani and Hamilton (1998), Ann. Allergy, Asthma & Immunol. 1: 105; Harris (1995) Biochem. Soc. Transactions 23: 1035; Hurle and Gross (1994) Curr. Op. Biotech. 5: 428; and U.S. Pat. Nos. 6,982,321 and 7,087,409.

“Immunoglobulin” (Ig) is used interchangeably with “antibody” herein. The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intra-chain disulfide bridges. Each H chain has, at the N-terminus, a variable domain (V_{H}) followed by three constant domains (C_{H}) for each of the α and γ chains and four C_{H} domains for p and E isotypes. Each L chain has at the N-terminus, a variable domain (V_{L}) followed by a constant domain at its other end. The V_{L} is aligned with the V_{H} and the CL is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8^{th} Edition, Sties et al. (eds.), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG and IgM, having heavy chains designated α, δ, ∈, γ and μ, respectively. The γ and α classes are further divided into subclasses on the basis of relatively minor differences in the C_{H} sequence and function, e.g., humans express the following subclasses: IgG1, IgG2A, IgG2B, IgG3, IgG4, IgA1, and IgK1.

“Infusion” or “infusing” refers to the introduction of a drug-containing solution into the body through a vein for therapeutic purposes. Generally, this is achieved via an intravenous (IV) bag.

“In combination with” or “in conjunction with” refers to administration of one treatment modality in addition to another treatment modality. As such, “in combination with” or “in conjunction with” refers to administration of one treatment modality before, during, or after administration of the other treatment modality to the individual. As used herein, the term “in combination” with regard to administration of Compound 1 and an additional chemotherapeutic agent means that each of Compound 1, or a pharmaceutically acceptable salt thereof, and the additional chemotherapeutic agent are administered to the patient in any order (i.e., simultaneously or sequentially) or together in a single composition, formulation or unit dosage form. In some embodiments, the term “combination” means that the Compound 1, or pharmaceutically acceptable salt thereof, and the additional therapeutic agent, are administered simultaneously or sequentially. In certain embodiments, the Compound 1, or pharmaceutically acceptable salt thereof, and the additional therapeutic agent, are administered simultaneously in the same composition comprising the Compound 1, or pharmaceutically acceptable salt thereof, and the additional therapeutic agent. In certain embodiments, the Compound 1, or pharmaceutically acceptable salt thereof, and the additional therapeutic agent, are administered simultaneously in separate compositions, i.e., wherein the Compound 1, or pharmaceutically acceptable salt thereof, and the additional therapeutic agent are administered simultaneously each in a separate unit dosage form. It will be appreciated that Compound 1, or a pharmaceutically acceptable salt thereof, and the additional chemotherapeutic agent are administered on the same day or on different days and in any order as according to an appropriate dosing protocol.

“Isolated” refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues. For example, an “isolated antibody” is one that has been identified, separated and/or recovered from a component of its production environment (e.g., natural or recombinant). Preferably, the isolated polypeptide is free of association with all other components from its production environment. Contaminant components of its production environment, such as that resulting from recombinant transfected cells, are materials that would typically interfere with research, diagnostic or therapeutic uses for the antibody, and may include enzymes, hormones, and other proteinaceous or non-proteinaceous solutes. In preferred embodiments, the polypeptide will be purified: (1) to greater than 95% by weight of antibody as determined by, for example, the Lowry method, and in some embodiments, to greater than 99% by weight; (1) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. The “isolated antibody” includes the antibody in-situ within recombinant cells since at least one component of the antibody's natural environment will not be present. Ordinarily, however, an isolated polypeptide or antibody will be prepared by at least one purification step.

“Metastatic” cancer refers to cancer which has spread from one part of the body (e.g., the lung) to another part of the body.

“Monoclonal antibody”, as used herein, refers to an antibody obtained from a population of substantially homogeneous antibodies, i.e., the individual antibodies comprising the population are identical except for possible naturally occurring mutations and/or post-translation modifications (e.g., isomerizations and amidations) that may be present in minor amounts. Monoclonal antibodies are highly specific, being directed against a single antigenic site. In contrast to polyclonal antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture and uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein (1975) Nature 256: 495; Hongo et al. (1995) Hybridoma 14 (3): 253; Harlow et al. (1988) Antibodies: A Laboratory Manual (Cold Spring Harbor Laboratory Press, 2^{nd} ed.; Hammerling et al. (1981) In: Monoclonal Antibodies and T-Cell Hybridomas 563 (Elsevier, N.Y.), recombinant DNA methods (see e.g., U.S. Pat. No. 4,816,567), phage-display technologies (see e.g., Clackson et al. (1991) Nature 352: 624; Marks et al. (1992) JMB 222: 581; Sidhu et al. (2004) JMB 338(2): 299; Lee et al. (2004) JMB 340(5): 1073; Fellouse (2004) PNAS USA 101(34): 12467; and Lee et al. (2004) J. Immunol. Methods 284(1-2): 119), and technologies for producing human or human-like antibodies in animals that have parts or all of the human immunoglobulin loci or genes encoding human immunoglobulin sequences (see e.g., WO 1998/24893; WO 1996/34096; WO 1996/33735; WO 1991/10741; Jakobovits et al. (1993) PNAS USA 90: 2551; Jakobovits et al. (1993) Nature 362: 255; Bruggemann et al. (1993) Year in Immunol. 7: 33; U.S. Pat. Nos. 5,545,807; 5,545,806; 5,569,825; 5,625,126; 5,633,425; and U.S. Pat. No. 5,661,016; Marks et al. (1992) Bio/Technology 10: 779; Lonberg et al. (1994) Nature 368: 856; Morrison (1994) Nature 368: 812; Fishwild et al. (1996) Nature Biotechnol. 14: 845; Neuberger (1996), Nature Biotechnol. 14: 826; and Lonberg and Huszar (1995), Intern. Rev. Immunol. 13: 65-93). The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (see e.g., U.S. Pat. No. 4,816,567; Morrison et al. (1984) PNAS USA, 81: 6851).

“Nanobodies” refer to single-domain antibodies, which are fragments consisting of a single monomeric variable antibody domain. Like a whole antibody, they are able to bind selectively to a specific antigen. With a molecular weight of only 12-15 kDa, single-domain antibodies are much smaller than common antibodies (150-160 kDa). The first single-domain antibodies were engineered from heavy-chain antibodies found in camelids (see e.g., W. Wayt Gibbs, “Nanobodies”, Scientific American Magazine (August 2005)).

“Objective response” refers to a measurable response, including complete response (CR) or partial response (PR).

“Partial response” refers to a decrease in the size of one or more tumors or lesions, or in the extent of cancer in the body, in response to treatment.

“Patient” and “subject” are used interchangeably herein to refer to a mammal in need of treatment for a cancer. Generally, the patient is a human diagnosed or at risk for suffering from one or more symptoms of a cancer. In certain embodiments a “patient” or “subject” may refer to a non-human mammal, such as a non-human primate, a dog, cat, rabbit, pig, mouse, or rat, or animals used in screening, characterizing, and evaluating drugs and therapies.

“PD-L1 expression” as used herein means any detectable level of expression of PD-L1 protein on the cell surface or of PD-L1 mRNA within a cell or tissue. PD-L1 protein expression may be detected with a diagnostic PD-L1 antibody in an IHC assay of a tumor tissue section or by flow cytometry. Alternatively, PD-L1 protein expression by tumor cells may be detected by PET imaging, using a binding agent (e.g., antibody fragment, affibody and the like) that specifically binds to PD-L1. Techniques for detecting and measuring PD-L1 mRNA expression include RT-PCR and real-time quantitative RT-PCR.

“PD-L1 positive” cancer, including a “PD-L1 positive” cancerous disease, is one comprising cells, which have PD-L1 present at their cell surface. The term “PD-L1 positive” also refers to a cancer that produces sufficient levels of PD-L1 at the surface of cells thereof, such that an anti-PD-L1 antibody has a therapeutic effect, mediated by the binding of the said anti-PD-L1 antibody to PD-L1.

“Pharmaceutically acceptable” indicates that the substance or composition must be compatible chemically and/or toxicologically, with the other ingredients comprising a formulation, and/or the mammal being treated therewith. “Pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof.

“Recurrent” cancer is one which has regrown, either at the initial site or at a distant site, after a response to initial therapy, such as surgery. A locally “recurrent” cancer is cancer that returns after treatment in the same place as a previously treated cancer.

“Reduction” of a symptom or symptoms (and grammatical equivalents of this phrase) refers to decreasing the severity or frequency of the symptom(s), or elimination of the symptom(s).

“Serum” refers to the clear liquid that can be separated from clotted blood. Serum differs from plasma, the liquid portion of normal unclotted blood containing the red and white cells and platelets. Serum is the component that is neither a blood cell (serum does not contain white or red blood cells) nor a clotting factor. It is the blood plasma not including the fibrinogens that help in the formation of blood clots. It is the clot that makes the difference between serum and plasma.

“Single-chain Fv”, also abbreviated as “sFv” or “scFv”, are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see e.g., Pluckthun (1994), In: The Pharmacology of Monoclonal Antibodies, vol. 113, Rosenburg and Moore (eds.), Springer-Verlag, New York, pp. 269.

“Suitable for therapy” or “suitable for treatment” shall mean that the patient is likely to exhibit one or more desirable clinical outcomes as compared to patients having the same cancer and receiving the same therapy but possessing a different characteristic that is under consideration for the purpose of the comparison. In one aspect, the characteristic under consideration is a genetic polymorphism or a somatic mutation (see e.g., Samsami et al. (2009) J Reproductive Med 54(1): 25). In another aspect, the characteristic under consideration is the expression level of a gene or a polypeptide. In one aspect, a more desirable clinical outcome is relatively higher likelihood of or relatively better tumor response such as tumor load reduction. In another aspect, a more desirable clinical outcome is relatively longer overall survival. In yet another aspect, a more desirable clinical outcome is relatively longer progression free survival or time to tumor progression. In yet another aspect, a more desirable clinical outcome is relatively longer disease free survival. In another aspect, a more desirable clinical outcome is relative reduction or delay in tumor recurrence. In another aspect, a more desirable clinical outcome is relatively decreased metastasis. In another aspect, a more desirable clinical outcome is relatively lower relative risk. In yet another aspect, a more desirable clinical outcome is relatively reduced toxicity or side effects. In some embodiments, more than one clinical outcomes are considered simultaneously. In one such aspect, a patient possessing a characteristic, such as a genotype of a genetic polymorphism, may exhibit more than one more desirable clinical outcomes as compared to patients having the same cancer and receiving the same therapy but not possessing the characteristic. As defined herein, the patient is considered suitable for the therapy. In another such aspect, a patient possessing a characteristic may exhibit one or more desirable clinical outcomes but simultaneously exhibit one or more less desirable clinical outcomes. The clinical outcomes will then be considered collectively, and a decision as to whether the patient is suitable for the therapy will be made accordingly, taking into account the patient's specific situation and the relevance of the clinical outcomes. In some embodiments, progression free survival or overall survival is weighted more heavily than tumor response in a collective decision making.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Systemic” treatment is a treatment, in which the drug substance travels through the bloodstream, reaching and affecting cells all over the body.

“Therapeutically effective amount” of an anti-PD-L1 antibody or antigen-binding fragment thereof, or a DNA-PK inhibitor, in each case of the invention, refers to an amount effective, at dosages and for periods of time necessary, that, when administered to a patient with a cancer, will have the intended therapeutic effect, e.g., alleviation, amelioration, palliation, or elimination of one or more manifestations of the cancer in the patient, or any other clinical result in the course of treating a cancer patient. A therapeutic effect does not necessarily occur by administration of one dose, and may occur only after administration of a series of doses. Thus, a therapeutically effective amount may be administered in one or more administrations. Such therapeutically effective amount may vary according to factors such as the disease state, age, sex, and weight of the individual, and the ability of an anti-PD-L1 antibody or antigen-binding fragment thereof, or a DNA-PK inhibitor, to elicit a desired response in the individual. A therapeutically effective amount is also one in which any toxic or detrimental effects of an anti-PD-L1 antibody or antigen-binding fragment thereof, or a DNA-PK inhibitor, are outweighed by the therapeutically beneficial effects.

“Treating” or “treatment of” a condition or patient refers to taking steps to obtain beneficial or desired results, including clinical results. For purposes of this invention, beneficial or desired clinical results include, but are not limited to, alleviation, amelioration of one or more symptoms of a cancer; diminishment of extent of disease; delay or slowing of disease progression; amelioration, palliation, or stabilization of the disease state; or other beneficial results. It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

“Variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al. (1991) Sequences of Immunological Interest, 5^{th} edition, National Institute of Health, Bethesda, Md.). The constant domains are not involved directly in the binding of antibody to an antigen, but exhibit various effector functions, such as participation of the antibody in antibody-dependent cellular toxicity.

“Variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

### Abbreviations

Some abbreviations used in the description include:
1 L: First line
2 L: Second line
ADCC: Antibody-dependent cell-mediated cytotoxicity
BID: Twice daily
CDR: Complementarity determining region
CR: Complete response
CRC: Colorectal cancer
CRT: Chemoradiotherapy
CT: Chemotherapy
DNA: Deoxyribonucleic acid
DNA-PK: DNA-dependent protein kinase
DNA-PKi: DNA-dependent protein kinase inhibitor
DSB: Double strand break
ED: Extensive disease
Eto: Etoposide
Ig: Immunoglobulin
IHC: Immunohistochemistry
IV: Intravenous
mCRC: Metastatic colorectal cancer
MSI-H: Microsatellite status instable high
MSI-L: Microsatellite status instable low
MSS: Microsatellite status stable
NK: Natural killers
NSCLC: Non-small-cell lung cancer
OS: Overall survival
PD: Progressive disease
PD-1: Programmed death 1
PD-L1: Programmed death ligand 1
PES: Polyester sulfone
PFS: Progression free survival
PR: Partial response
QD: Once daily
QID: Four times a day
Q2W: Every two weeks
Q3W: Every three weeks
RNA: Ribonucleic acid
RP2D: Recommended phase II dose
RR: Relative risk
RT: Radiotherapy
SCCHN: Squamous cell carcinoma of the head and neck
SCLC: Small-cell lung cancer
SoC: Standard of care
SR: Sustained response
TID: Three times a day
Topo: Topotecan
TR: Tumor response
TTP: Time to tumor progression
TTR: Time to tumor recurrence

### DESCRIPTIVE EMBODIMENTS

Therapeutic Combination and Method of Use Thereof

Some chemotherapies and radiotherapy can promote immunogenic tumor cell death and shape the tumor microenvironment to promote antitumor immunity. DNA-PK inhibition by means of DNA repair inhibitors can trigger and increase the immunogenic cell death induced by radiotherapy or chemotherapy and may therefore further increase T cell responses. The activation of the stimulator of interferon genes (STING) pathway and subsequent induction of type I interferons and PD-L1 expression is part of the response to double strand breaks in the DNA. Further, tumors with high somatic mutation burden are particularly responsive to checkpoint inhibitors, potentially due to increased neo-antigen formation. Particularly, there is a strong anti-PD1 response in mismatch repair-deficient CRC. DNA repair inhibitors may further increase the mutation rate of tumors and thus the repertoire of neo-antigens. Without being bound by any theory, the inventors assume that gathering double strand breaks (DSBs), e.g., by inhibiting DSB repair, particularly in combination with DNA-damaging interventions such as radiotherapy or chemotherapy, or in genetically instable tumors, sensitizes tumors to the treatment with an anti-PD-L1 antibody comprising a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. Inhibition of the interaction between PD-1 and PD-L1 enhances T-cell responses and mediates clinical antitumor activity. PD-1 is a key immune checkpoint receptor expressed by activated T cells, which mediates immunosuppression and functions primarily in peripheral tissues, where T cells may encounter the immunosuppressive PD-1 ligands PD-L1 (B7-H1) and PD-L2 (B7-DC), which are expressed by tumor cells, stromal cells, or both.

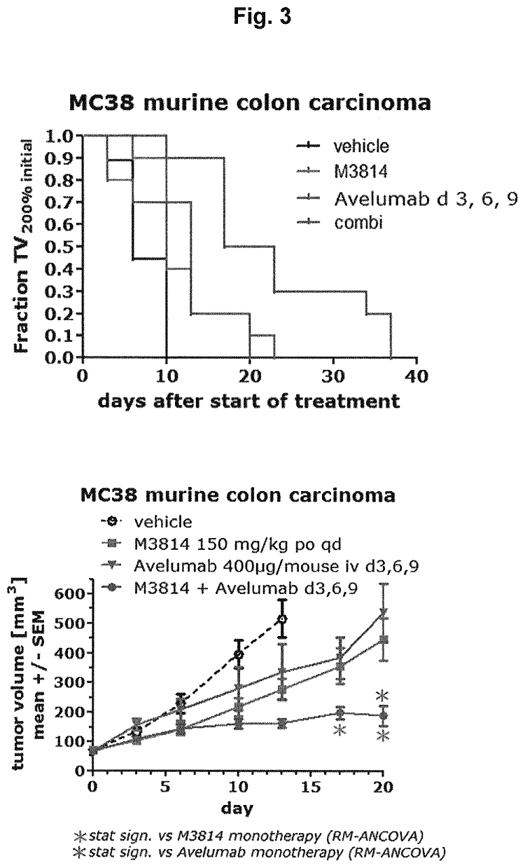

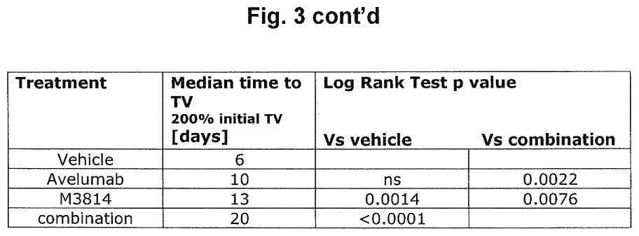

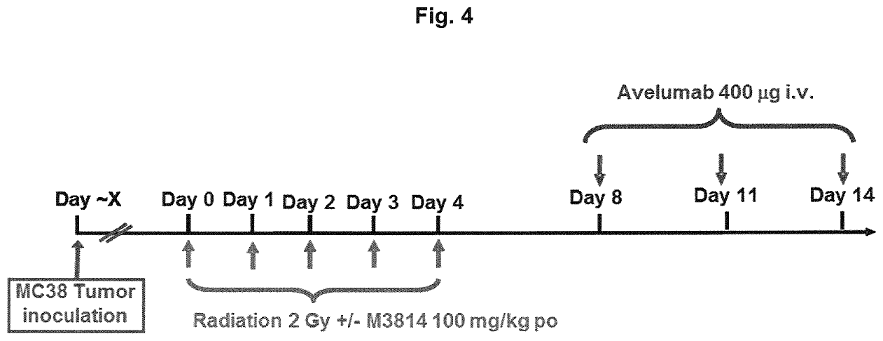

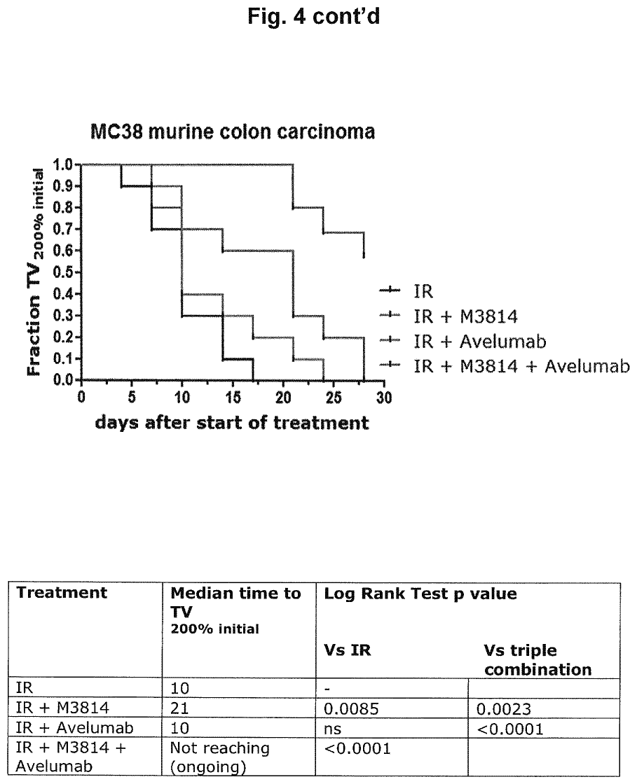

The present invention arose in part from the surprising discovery of a combination benefit for a DNA-PK inhibitor and an anti-PD-L1 antibody, as well as for a DNA-PK inhibitor and an anti-PD-L1 antibody in combination with radiotherapy, chemotherapy or chemoradiotherapy, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. Adding a DNA-PK inhibitor to the said anti-PD-L1 antibody was expected to be contraindicated, since DNA-PK is a major enzyme in VDJ recombination and as such potentially immunosuppressive to such an extent that deletion of DNA-PK leads to the SCID (severe combined immune deficiency) phenotype in mice. In contrast, the combination of the present invention delayed the tumor growth as compared to the single agent treatment (see e.g., FIG. 3). Treatment schedule and doses were designed to reveal potential synergies. Optimal Compound 1/radiotherapy regimens as well as avelumab/radiotherapy regimens would be too efficacious in this particular tumor model. In-vitro data demonstrated a synergy of the DNA-PK inhibitor, particularly Compound 1, in combination with the PD-L1 antibody, particularly avelumab, optionally together with radiotherapy, versus the DNA-PK inhibitor or avelumab (see e.g., FIG. 3 or 4).

Thus, in one aspect, the present invention provides a method for treating a cancer in a subject in need thereof, comprising administering to the subject an anti-PD-L1 antibody, or an antigen-binding fragment or functional fragment thereof, and a DNA-PK inhibitor. It shall be understood that a therapeutically effective amount of the anti-PD-L1 antibody and DNA-PK inhibitor is applied in the method of the invention, which is sufficient for treating one or more symptoms of a disease or disorder associated with PD-L1 and DNA-PK, respectively.

Particularly, the present invention provides a method for treating a cancer in a subject in need thereof, comprising administering to the subject an anti-PD-L1 antibody, or an antigen-binding fragment thereof, and a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

In one embodiment, the anti-PD-L1 antibody is a monoclonal antibody. In one embodiment, the anti-PD-L1 antibody exerts antibody-dependent cell-mediated cytotoxicity (ADCC). In one embodiment, the anti-PD-L1 antibody is a human or humanized antibody. In one embodiment, the anti-PD-L1 antibody is an isolated antibody. In various embodiments, the anti-PD-L1 antibody is characterized by a combination of one or more of the foregoing features, as defined above.

In various embodiments, the anti-PD-L1 antibody is avelumab. Avelumab (formerly designated MSB0010718C) is a fully human monoclonal antibody of the immunoglobulin (Ig) G1 isotype (see e.g., WO 2013/079174). Avelumab selectively binds to PD-L1 and competitively blocks its interaction with PD-1. The mechanisms of action rely on the inhibition of PD-1/PD-L1 interaction and on natural killer (NK)-based antibody-dependent cell-mediated cytotoxicity (ADCC) (see e.g., Boyerinas et al. (2015) Cancer Immunol Res 3: 1148). Compared with anti-PD-1 antibodies that target T cells, avelumab targets tumor cells and therefore, it is expected to have fewer side effects, including a lower risk of autoimmune-related safety issues, as the blockade of PD-L1 leaves the PD-L2/PD-1 pathway intact to promote peripheral self-tolerance (see e.g., Latchman et al. (2001) Nat Immunol 2(3): 261).

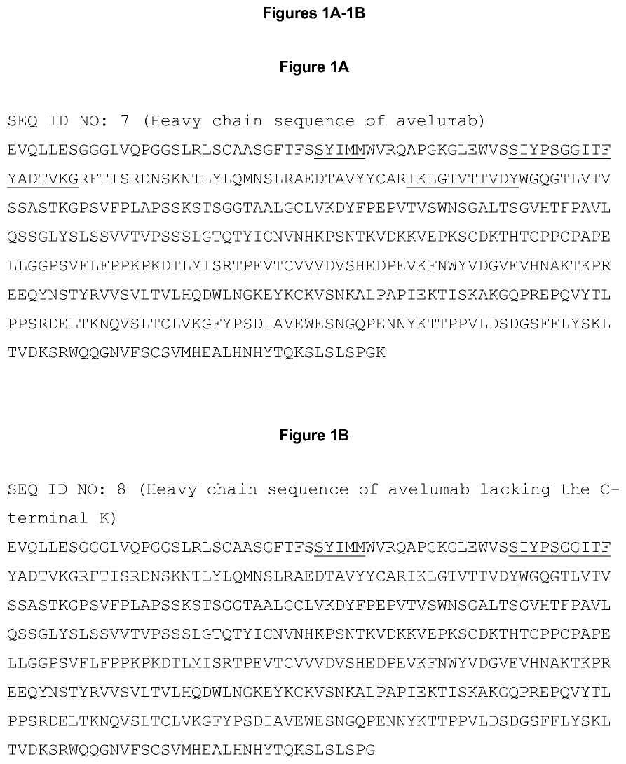

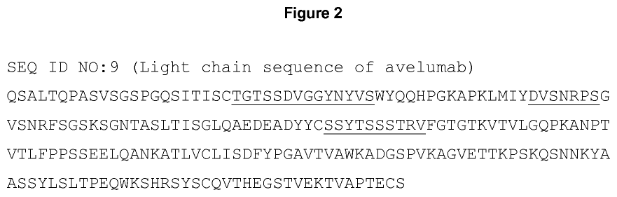

Avelumab, its sequence, and many of its properties have been described in WO 2013/079174, where it is designated A09-246-2 having the heavy and light chain sequences according to SEQ ID NOs: 32 and 33, as shown in FIG. 1 (SEQ ID NO: 7) and FIG. 2 (SEQ ID NO: 9), of this patent application. It is frequently observed, however, that in the course of antibody production the C-terminal lysine (K) of the heavy chain is cleaved off. This modification has no influence on the antibody-antigen binding. Therefore, in some embodiments the C-terminal lysine (K) of the heavy chain sequence of avelumab is absent. The heavy chain sequence of avelumab without the C-terminal lysine is shown in FIG. 1B (SEQ ID NO: 8), whereas FIG. 1A (SEQ ID NO: 7) shows the full length heavy chain sequence of avelumab. Further, as shown in WO 2013/079174, one of avelumab's properties is its ability to exert antibody-dependent cell-mediated cytotoxicity (ADCC), thereby directly acting on PD-L1 bearing tumor cells by inducing their lysis without showing any significant toxicity. In a preferred embodiment, the anti-PD-L1 antibody is avelumab, having the heavy and light chain sequences shown in FIG. 1A or 1B (SEQ ID NOs: 7 or 8), and FIG. 2 (SEQ ID NO: 9), or an antigen-binding fragment thereof.

In some aspects, the DNA-PK inhibitor is (S)-[2-chloro-4-fluoro-5-(7-morpholin-4-yl-quinazolin-4-yl)-phenyl]-(6-methoxypyridazin-3-yl)-methanol, having the structure of Compound 1:

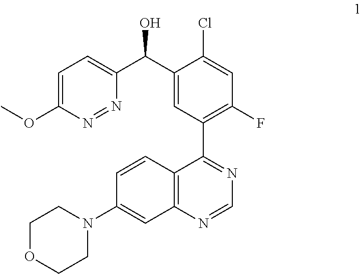 [SMILES: COc1ccc([C@@H](O)c2cc(-c3ncnc4cc(N5CCOCC5)ccc34)c(F)cc2Cl)nn1]

or a pharmaceutically acceptable salt thereof.

Compound 1 is described in detail in United States patent application US 2016/0083401, published on Mar. 24, 2016 (referred to herein as “the '401 publication”), the entirety of which is hereby incorporated herein by reference. Compound 1 is designated as compound 136 in Table 4 of the '401 publication. Compound 1 is active in a variety of assays and therapeutic models demonstrating inhibition of DNA-PK (see, e.g., Table 4 of the '401 publication). Accordingly, Compound 1, or a pharmaceutically acceptable salt thereof, is useful for treating one or more disorders associated with activity of DNA-PK, as described in detail herein.

Compound 1 is a potent and selective ATP-competitive inhibitor of DNA-PK, as demonstrated by crystallographic and enzyme kinetics studies. DNA-PK, together with five additional protein factors (Ku70, Ku80, XRCC4, Ligase IV and Artemis) plays a critical role in the repair of DSB via NHEJ. Kinase activity of DNA-PK is essential for proper and timely DNA repair and the long-term survival of cancer cells. Without wishing to be bound by any particular theory, it is believed that the primary effects of Compound 1 are suppression of DNA-PK activity and DNA double strand break (DSB) repair, leading to altered repair of DNA and potentiation of antitumor activity of DNA-damaging agents.

It is understood that although the methods described herein may refer to formulations, doses and dosing regimens/schedules of Compound 1, such formulations, doses and/or dosing regimens/schedules are equally applicable to any pharmaceutically acceptable salt of Compound 1. Accordingly, in some embodiments, a dose or dosing regimen for a pharmaceutically acceptable salt of Compound 1, or a pharmaceutically acceptable salt thereof, is selected from any of the doses or dosing regimens for Compound 1 as described herein.

A pharmaceutically acceptable salt may involve the inclusion of another molecule, such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. If the compound of the invention is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In one embodiment, the therapeutic combination of the invention is used in the treatment of a human subject. In one embodiment, the anti-PD-L1 antibody targets PD-L1 which is human PD-L1. The main expected benefit in the treatment with the therapeutic combination is a gain in risk/benefit ratio with said antibody, particularly avelumab, for these human patients.

In one embodiment, the cancer is identified as a PD-L1 positive cancerous disease. Pharmacodynamic analyses show that tumor expression of PD-L1 might be predictive of treatment efficacy. According to the invention, the cancer is preferably considered to be PD-L1 positive if between at least 0.1% and at least 10% of the cells of the cancer have PD-L1 present at their cell surface, more preferably between at least 0.5% and 5%, most preferably at least 1%. In one embodiment, the PD-L1 expression is determined by immunohistochemistry (IHC).

In another embodiment, the cancer is selected from cancer of the lung, head and neck, colon, neuroendocrine system, mesenchyme, breast, ovarian, pancreatic, and histological subtypes thereof (e.g., adeno, squamous, large cell). In a preferred embodiment, the cancer is selected from small-cell lung cancer (SCLC), non-small-cell lung cancer (NSCLC), squamous cell carcinoma of the head and neck (SCCHN), colorectal cancer (CRC), primary neuroendocrine tumors and sarcoma.

In various embodiments, the method of the invention is employed as a first, second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

If the safety and the clinical benefit offered by the therapeutic combination of the invention are confirmed, this combination of an anti-PD-L1 antibody and a DNA-PK inhibitor warrants a first-line setting in cancer patients. Particularly, the combination may become a new standard treatment for patients suffering from a cancer that is selected from the group of SCLC extensive disease (ED), NSCLC and SCCHN.

It is preferred that the therapeutic combination of the invention is applied in a later line of treatment, particularly a second-line or higher treatment of the cancer. There is no limitation to the prior number of therapies provided that the subject underwent at least one round of prior cancer therapy. The round of prior cancer therapy refers to a defined schedule/phase for treating a subject with, e.g., one or more chemotherapeutic agents, radiotherapy or chemoradiotherapy, and the subject failed with such previous treatment, which was either completed or terminated ahead of schedule. One reason could be that the cancer was resistant or became resistant to prior therapy. The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of an anti-PD-L1 antibody/DNA-PK inhibitor combination, preferably avelumab and (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof, seems to be much better than the SoC chemotherapy. In one embodiment, an anti-PD-L1 antibody/DNA-PK inhibitor combination, preferably avelumab and (S)-[2-chloro-4-fluoro-5-(7-morpholin-4-yl-quinazolin-4-yl)-phenyl]-(6-methoxypyridazin-3-yl)-methanol, or a pharmaceutically acceptable salt thereof, may be as effective and better tolerated than SoC chemotherapy in patients with cancer resistant to mono- and/or poly-chemotherapy, radiotherapy or chemoradiotherapy.

In a preferred embodiment, the anti-PD-L1 antibody and DNA-PK inhibitor are administered in a second-line or higher treatment, more preferably a second-line treatment, of the cancer selected from the group of pre-treated relapsing metastatic NSCLC, unresectable locally advanced NSCLC, pre-treated SCLC ED, SCLC unsuitable for systemic treatment, pre-treated relapsing (recurrent) or metastatic SCCHN, recurrent SCCHN eligible for re-irradiation, and pre-treated microsatellite status instable low (MSI-L) or microsatellite status stable (MSS) metastatic colorectal cancer (mCRC). SCLC and SCCHN are particularly systemically pre-treated. MSI-L/MSS mCRC occurs in 85% of all mCRC. Once, the safety/tolerability and efficacy profile of the dual combination of anti-PD-L1 antibody and DNA-PK inhibitor is established in patients, using the standard dose of the anti-PD-L1 antibody and the recommended phase II dose (RP2D) of the DNA-PK inhibitor, in each case as described herein, additional expansion cohorts including chemotherapy (e.g., etoposide or topotecan), radiotherapy or chemoradiotherapy to introduce double-strand breaks are targeted.

In some embodiments that employ an anti-PD-L1 antibody in the combination therapy, the dosing regimen will comprise administering the anti-PD-L1 antibody at a dose of about 1, 2, 3, 4, 5, 6, 7, 8, 9, 10, 11, 12, 13, 14, 15, 16, 17, 18, 19 or 20 mg/kg at intervals of about 14 days (±2 days) or about 21 days (±2 days) or about 30 days (±2 days) throughout the course of treatment. In other embodiments that employ an anti-PD-L1 antibody in the combination therapy, the dosing regimen will comprise administering the anti-PD-L1 antibody at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose. In certain embodiments, a subject will be administered an intravenous (IV) infusion of a medicament comprising any of the anti-PD-L1 antibody described herein. In some embodiments, the anti-PD-L1 antibody in the combination therapy is avelumab, which is administered intravenously at a dose selected from the group consisting of: about 1 mg/kg Q2W (Q2W=one dose every two weeks), about 2 mg/kg Q2W, about 3 mg/kg Q2W, about 5 mg/kg Q2W, about 10 mg/kg Q2W, about 1 mg/kg Q3W (Q3W=one dose every three weeks), about 2 mg/kg Q3W, about 3 mg/kg Q3W, about 5 mg/kg Q3W, and about 10 mg Q3W. In some embodiments of the invention, the anti-PD-L1 antibody in the combination therapy is avelumab, which is administered in a liquid medicament at a dose selected from the group consisting of about 1 mg/kg Q2W, about 2 mg/kg Q2W, about 3 mg/kg Q2W, about 5 mg/kg Q2W, about 10 mg/kg Q2W, about 1 mg/kg Q3W, about 2 mg/kg Q3W, about 3 mg/kg Q3W, about 5 mg/kg Q3W, and about 10 mg/kg Q3W. In some embodiments, a treatment cycle begins with the first day of combination treatment and last for 2 weeks. In such embodiments, the combination therapy is preferably administered for at least 12 weeks (6 cycles of treatment), more preferably at least 24 weeks, and even more preferably at least 2 weeks after the patient achieves a CR.

In some embodiments that employ an anti-PD-L1 antibody in the combination therapy, the dosing regimen will comprise administering the anti-PD-L1 antibody at a dose of about 400-800 mg flat dose Q2W. Preferably, the flat dosing regimen is 400 mg, 450 mg, 500 mg, 550 mg, 600 mg, 650 mg, 700 mg 750 mg or 800 mg flat dose Q2W. More preferably, the flat dosing regimen is 800 mg flat dose Q2W. In some more preferred embodiments that employ an anti-PD-L1 antibody in the combination therapy, the dosing regimen will be a fixed dose of 800 mg given intravenously at intervals of about 14 days (±2 days).

In another embodiment, the anti-PD-L1 antibody, preferably avelumab, will be given IV every two weeks (Q2W). In certain embodiments, the anti-PD-L1 antibody is administered intravenously for 50-80 minutes at a dose of about 10 mg/kg body weight every two weeks (Q2W). In a more preferred embodiment, the avelumab dose will be 10 mg/kg body weight administered as 1-hour intravenous infusions every two weeks (Q2W). In certain embodiments, the anti-PD-L1 antibody is administered intravenously for 50-80 minutes at a fixed dose of about 800 mg every two weeks (Q2W). In a more preferred embodiment, the avelumab dose will be 800 mg administered as 1-hour intravenous infusions every 2 weeks (Q2W). Given the variability of infusion pumps from site to site, a time window of minus 10 minutes and plus 20 minutes is permitted.

Pharmacokinetic studies demonstrated that the 10 mg/kg dose of avelumab achieves excellent receptor occupancy with a predictable pharmacokinetics profile (see e.g., Heery et al. (2015) Proc 2015 ASCO Annual Meeting, abstract 3055). This dose is well tolerated, and signs of antitumor activity, including durable responses, have been observed. Avelumab may be administered up to 3 days before or after the scheduled day of administration of each cycle due to administrative reasons. Pharmacokinetic simulations also suggested that exposures to avelumab across the available range of body weights are less variable with 800 mg Q2W compared with 10 mg/kg Q2W. Exposures were similar near the population median weight.

Low-weight subjects tended towards marginally lower exposures relative to the rest of the population when weight based dosing was used, and marginally higher exposures when flat dosing was applied. The implications of these exposure differences are not expected to be clinically meaningful at any weight across the whole population. Furthermore, the 800 mg Q2W dosing regimen is expected to result in C_{trough}>1 mg/mL required to maintain avelumab serum concentrations at >95% TO throughout the entire Q2W dosing interval in all weight categories. In a preferred embodiment, a fixed dosing regimen of 800 mg administered as a 1 hour IV infusion Q2W will be utilized for avelumab in clinical trials.

In some embodiments, provided methods comprise administering a pharmaceutically acceptable composition comprising the DNA-PK inhibitor, preferably Compound 1, or a pharmaceutically acceptable salt thereof, one, two, three or four times a day. In some embodiments, a pharmaceutically acceptable composition comprising the DNA-PK inhibitor, preferably Compound 1, or a pharmaceutically acceptable salt thereof, is administered once daily (“QD”), particularly continuously. In some embodiments, a pharmaceutically acceptable composition comprising the DNA-PK inhibitor, preferably Compound 1, or a pharmaceutically acceptable salt thereof, is administered twice daily, particularly continuously. In some embodiments, twice daily administration refers to a compound or composition that is administered “BID”, or two equivalent doses administered at two different times in one day. In some embodiments, a pharmaceutically acceptable composition comprising the DNA-PK inhibitor, preferably Compound 1, or a pharmaceutically acceptable salt thereof, is administered three times a day. In some embodiments, a pharmaceutically acceptable composition comprising Compound 1, or a pharmaceutically acceptable salt thereof, is administered “TID”, or three equivalent doses administered at three different times in one day. In some embodiments, a pharmaceutically acceptable composition comprising the DNA-PK inhibitor, preferably Compound 1, or a pharmaceutically acceptable salt thereof, is administered four times a day. In some embodiments, a pharmaceutically acceptable composition comprising Compound 1, or a pharmaceutically acceptable salt thereof, is administered “QID”, or four equivalent doses administered at four different times in one day. In some embodiments, the DNA-PK inhibitor, preferably Compound 1, or a pharmaceutically acceptable salt thereof, is administered to a patient under fasted conditions and the total daily dose is any of those contemplated above and herein. In some embodiments, the DNA-PK inhibitor, preferably Compound 1, or a pharmaceutically acceptable salt thereof, is administered to a patient under fed conditions and the total daily dose is any of those contemplated above and herein. In some embodiments, the DNA-PK inhibitor, preferably Compound 1, or a pharmaceutically acceptable salt thereof, is administered orally. In some embodiments, the DNA-PK inhibitor, preferably Compound 1, or a pharmaceutically acceptable salt thereof, will be given orally either once or twice daily continuously. In preferred embodiments, the DNA-PK inhibitor, preferably Compound 1, or a pharmaceutically acceptable salt thereof, is administered once daily (QD) or twice daily (BID), at a dose of about 1 to about 800 mg. In preferred embodiments, the DNA-PK inhibitor, preferably Compound 1, or a pharmaceutically acceptable salt thereof, is administered twice daily (BID), at a dose of about 400 mg.

Concurrent treatment considered necessary for the patient's well-being may be given at discretion of the treating physician. In some embodiments, the anti-PD-L1 antibody and DNA-PK inhibitor are administered in combination with chemotherapy (CT), radiotherapy (RT), or chemotherapy and radiotherapy (CRT). As described herein, in some embodiments, the present invention provides methods of treating, stabilizing or decreasing the severity or progression of one or more diseases or disorders associated with PD-L1 and DNA-PK comprising administering to a patient in need thereof an anti-PD-L1 antibody and an inhibitor of DNA-PK in combination with an additional chemotherapeutic agent. In certain embodiments, the chemotherapeutic agent is selected from the group of etoposide, doxorubicin, topotecan, irinotecan, fluorouracil, a platin, an anthracycline, and a combination thereof.

In certain embodiments, the additional chemotherapeutic agent is etoposide. Etoposide forms a ternary complex with DNA and the topoisomerase II enzyme which aids in DNA unwinding during replication. This prevents re-ligation of the DNA strands and causes DNA strands to break. Cancer cells rely on this enzyme more than healthy cells because they divide more rapidly. Therefore, etoposide treatment causes errors in DNA synthesis and promotes apoptosis of the cancer cells. Without wishing to be bound by any particular theory, it is believed that a DNA-PK inhibitor blocks one of the main pathways for repair of DSBs in DNA thus delaying the repair process and leading to an enhancement of the antitumor activity of etoposide. In-vitro data demonstrated a synergy of Compound 1 in combination with etoposide versus etoposide alone. Thus, in some embodiments, a provided combination of Compound 1, or a pharmaceutically acceptable salt thereof, with etoposide is synergistic.

In certain embodiments, the additional chemotherapeutic agent is topotecan.

In certain embodiments, the therapeutic combination of the invention is combined further with chemotherapy, which is especially etoposide and antracycline treatment, either as single cytostatic agent or as part of a doublet or triplet regiment. With such a chemotherapy, the DNA-PK inhibitor can be preferably given once or twice daily with the anti-PD-L1 antibody, particularly avelumab, which is given every two weeks. In cases, in which anthracyclines are used, the treatment with anthracycline is stopped once a maximal life-long accumulative dose has been reached (due to the cardiotoxicity).

In certain embodiments, the additional chemotherapeutic agent is a platin. Platins are platinum-based chemotherapeutic agents. As used herein, the term “platin” is used interchangeably with the term “platinating agent.” Platinating agents are well known in the art. In some embodiments, the platin (or platinating agent) is selected from cisplatin, carboplatin, oxaliplatin, nedaplatin, and satraplatin.

In certain embodiments, the platin is cisplatin. Cisplatin crosslinks cellular DNA in several different ways interfering with cell division by mitosis. Most notable among the changes in DNA are the intra-strand cross-links with purine bases. These crosslinks are repaired primarily by nucleotide excision repair. The damaged DNA activates checkpoint mechanisms, which in turn activate apoptosis when repair proves impossible. In certain embodiments, the provided method further comprises administration of radiation therapy to the patient.

In certain embodiments, the additional chemotherapeutic agent is carboplatin.

In some embodiments, the additional chemotherapeutic is a combination of both of etoposide and a platin. In certain embodiments, the present invention provides a method of treating a cancer selected from lung, head and neck, colon, neuroendocrine system, mesenchyme, breast, ovarian, pancreatic, and histological subtypes thereof (e.g., adeno, squamous, large cell) in a patient in need thereof comprising administering to said patient the anti-PD-L1 antibody and DNA-PK inhibitor, preferably Compound 1 or a pharmaceutically acceptable salt thereof, in combination with at least one additional therapeutic agent selected from etoposide and a platin. In certain embodiments, the provided method further comprises administration of radiation therapy to the patient.

In some embodiments, the additional chemotherapeutic is a combination of both of etoposide and cisplatin. In certain embodiments, the present invention provides a method of treating a cancer selected from lung, head and neck, colon, neuroendocrine system, mesenchyme, breast, pancreatic, and histological subtypes thereof (e.g., adeno, squamous, large cell) in a patient in need thereof comprising administering to said patient the anti-PD-L1 antibody and DNA-PK inhibitor, preferably Compound 1 or a pharmaceutically acceptable salt thereof, in combination with at least one additional therapeutic agent selected from etoposide and cisplatin. In certain embodiments, the provided method further comprises administration of radiation therapy to the patient.

In some embodiments, the additional chemotherapeutic is a combination of both of etoposide and carboplatin.

The DNA-PK inhibitor, preferably Compound 1, or a pharmaceutically acceptable salt thereof, and compositions thereof in combination with the anti-PD-L1 antibody and additional chemotherapeutic according to methods of the present invention, are administered using any amount and any route of administration effective for treating or decreasing the severity of a disorder provided above. The exact amount required will vary from subject to subject, depending on the species, age, and general condition of the subject, the severity of the infection, the particular agent, its mode of administration, and the like.

In some embodiments, the present invention provides a method of treating a cancer selected from lung, head and neck, colon, neuroendocrine system, mesenchyme, breast, ovarian, pancreatic, and histological subtypes thereof (e.g., adeno, squamous, large cell) in a patient in need thereof comprising administering to said patient the DNA-PK inhibitor, preferably Compound 1, or a pharmaceutically acceptable salt thereof, in an amount of about 1 to about 800 mg, preferably in an amount of about 10 to about 800 mg, more preferably in an amount of about 100 to about 400 mg, in each case in combination with the anti-PD-L1 antibody and at least one additional therapeutic agent selected from a platin and etoposide, in amounts according to the local clinical standard of care guidelines.

In some embodiments, provided methods comprise administering a pharmaceutically acceptable composition comprising a chemotherapeutic agent one, two, three or four times a day. In some embodiments, a pharmaceutically acceptable composition comprising a chemotherapeutic agent is administered once daily (“QD”). In some embodiments, a pharmaceutically acceptable composition comprising a chemotherapeutic agent is administered twice daily. In some embodiments, twice daily administration refers to a compound or composition that is administered “BID”, or two equivalent doses administered at two different times in one day. In some embodiments, a pharmaceutically acceptable composition comprising a chemotherapeutic agent is administered three times a day. In some embodiments, a pharmaceutically acceptable composition comprising a chemotherapeutic agent is administered “TID”, or three equivalent doses administered at three different times in one day. In some embodiments, a pharmaceutically acceptable composition comprising a chemotherapeutic agent is administered four times a day. In some embodiments, a pharmaceutically acceptable composition comprising a chemotherapeutic agent is administered “QID”, or four equivalent doses administered at four different times in one day. In some embodiments, a pharmaceutically acceptable composition comprising a chemotherapeutic agent is administered for a various number of days (for example 14, 21, 28) with a various number of days between treatment (0, 14, 21, 28). In some embodiments, a chemotherapeutic agent is administered to a patient under fasted conditions and the total daily dose is any of those contemplated above and herein. In some embodiments, a chemotherapeutic agent is administered to a patient under fed conditions and the total daily dose is any of those contemplated above and herein. In some embodiments, a chemotherapeutic agent is administered orally for reasons of convenience. In some embodiments, when administered orally, a chemotherapeutic agent is administered with a meal and water. In another embodiment, the chemotherapeutic agent is dispersed in water or juice (e.g., apple juice or orange juice) and administered orally as a suspension. In some embodiments, when administered orally, a chemotherapeutic agent is administered in a fasted state. A chemotherapeutic agent can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In some embodiments, etoposide is administered via intravenous infusion. In some embodiments, etoposide is administered intravenously in an amount of about 50 to about 100 mg/m². Most commonly, etoposide is administered at 100 mg/m². In some embodiments, etoposide is administered via intravenous infusion over about 1 hour. In certain embodiments, the etoposide is administered via intravenous infusion at about 100 mg/m² over a 60-minute period. In some embodiments, etoposide is administered on day 1 to 3 every three weeks (D1-3Q3W), in an amount of about 100 mg/m². In certain embodiments, etoposide is administered via intravenous infusion on Day 1 and then via intravenous infusion or oral administration on Days 2 and 3.

In certain embodiments, topotecan is administered on day 1 to 5 every three weeks (D1-5 Q3W).

In certain embodiments, cisplatin is administered via intravenous infusion. In some embodiments, cisplatin is administered via intravenous infusion over about 1 hour. In certain embodiments, cisplatin is administered intravenously in an amount of about 50 to about 75 mg/m². Most commonly, cisplatin is administered at 75 mg/m². In certain embodiments, cisplatin is administered via intravenous infusion at about 75 mg/m² over a 60-minute period. In some embodiments, cisplatin is administered once every three weeks (Q3W), in an amount of about at 75 mg/m².

In certain embodiments, the present invention provides a method of treating a cancer in a patient in need thereof comprising administering to said patient the DNA-PK inhibitor, preferably Compound 1, or a pharmaceutically acceptable salt thereof, in combination with cisplatin and etoposide. Most commonly, cisplatin is administered at 75 mg/m² and etoposide at 100 mg/m².

In some embodiments, etoposide and cisplatin are administered sequentially in either order or substantially simultaneously. The additional chemotherapeutic agents are administered to the patient in any order (i.e., simultaneously or sequentially) in separate compositions, formulations or unit dosage forms, or together in a single composition, formulation or unit dosage form. In certain embodiments, etoposide is administered simultaneously in the same composition comprising etoposide and cisplatin. In certain embodiments, etoposide and cisplatin are administered simultaneously in separate compositions, i.e., wherein etoposide and cisplatin are administered simultaneously each in a separate unit dosage form. It will be appreciated that etoposide and cisplatin are administered on the same day or on different days and in any order as according to an appropriate dosing protocol.

In certain embodiments, the present invention provides a method of treating a cancer, preferably selected from lung, head and neck, colon, neuroendocrine system, mesenchyme, breast, ovarian, pancreatic, and histological subtypes thereof (e.g., adeno, squamous, large cell), in a patient in need thereof comprising administering to said patient the DNA-PK inhibitor, preferably Compound 1, or a pharmaceutically acceptable salt thereof, in combination with the anti-PD-L1 antibody and at least one additional therapeutic agent, preferably selected from etoposide and cisplatin, wherein (i) the DNA-PK inhibitor, preferably Compound 1, or a pharmaceutically acceptable salt thereof, and the additional therapeutic agent are provided in the same composition, optionally together with the anti-PD-L1 antibody, (ii) the DNA-PK inhibitor, preferably Compound 1, or a pharmaceutically acceptable salt thereof, and the anti-PD-L1 antibody are provided in the same composition, optionally together with the additional therapeutic agent, or (iii) the anti-PD-L1 antibody and the additional therapeutic agent are provided in the same composition, optionally together with the DNA-PK inhibitor, preferably Compound 1, or a pharmaceutically acceptable salt thereof. In certain embodiments, the provided method further comprises administration of radiation therapy to the patient.

In certain embodiments, the present invention provides a method of treating a cancer, preferably selected from lung, head and neck, colon, neuroendocrine system, mesenchyme, breast, pancreatic, and histological subtypes thereof (e.g., adeno, squamous, large cell), in a patient in need thereof comprising administering to said patient the DNA-PK inhibitor, preferably Compound 1, or a pharmaceutically acceptable salt thereof, in combination with the anti-PD-L1 antibody and at least one additional therapeutic agent, preferably selected from etoposide and cisplatin, wherein the DNA-PK inhibitor, preferably Compound 1, or a pharmaceutically acceptable salt thereof, the anti-PD-L1 antibody and the additional therapeutic agent are provided in separate compositions for simultaneous or sequential administration to said patient. In certain embodiments, the provided method further comprises administration of radiation therapy to the patient.

In some embodiments, the present invention provides a method of treating a cancer in a patient in need thereof comprising administering to said patient the DNA-PK inhibitor, preferably Compound 1, or a pharmaceutically acceptable salt thereof, followed by administration of cisplatin and then administration of etoposide. In certain embodiments, the DNA-PK inhibitor, preferably Compound 1, is administered about 1-2, preferably about 1.5 hours prior to administration of the cisplatin. In some embodiments, the DNA-PK inhibitor, preferably Compound 1, is administered to said patient QD. In certain embodiments, the DNA-PK inhibitor, preferably Compound 1, is administered for 5 days. In some embodiments, the DNA-PK inhibitor, preferably Compound 1, is administered from about 4 days to about 3 weeks, for about 5 days, for about 1 week, or for about 2 weeks.

In certain embodiments, the anti-PD-L1 antibody and DNA-PK inhibitor, preferably Compound 1, or a pharmaceutically acceptable salt thereof, are administered in combination with radiotherapy. In certain embodiments, provided methods comprise administration of the anti-PD-L1 antibody and DNA-PK inhibitor, preferably Compound 1, or a pharmaceutically acceptable salt thereof, in combination with one or both of etoposide and cisplatin, wherein said method further comprises administering radiotherapy to the patient. In certain embodiments, the radiotherapy comprises about 35-70 Gy/20-35 fractions. In some embodiments, the radiotherapy is given either with standard fractionation (1.8 to 2 Gy for day 5 days a week) up to a total dose of 50-70 Gy in once daily. Other fractionation schedules could also be envisioned, for example, a lower dose per fraction but given twice daily with the DNA-PK inhibitor given also twice daily. Higher daily doses over a shorter period of time can also be given. In one embodiment, stereotactic radiotherapy as well as the gamma knife are used. In the palliative setting, other fractionation schedules are also widely used for example 25 Gy in 5 fractions or 30 Gy in 10 fractions. In all cases, avelumab is preferably given every two weeks. For radiotherapy, the duration of treatment will be the time frame when radiotherapy is given. These interventions apply to treatment given with electrons, photons and protons, alfa-emitters or other ions, treatment with radio-nucleotides, for example, treatment with ¹³¹I given to patients with thyroid cancer, as well in patients treated with boron capture neutron therapy.

In some embodiments, the combination regimen comprises the steps of: (a) under the direction or control of a physician, the subject receiving the PD-L1 antibody prior to first receipt of the DNA-PK inhibitor; and (b) under the direction or control of a physician, the subject receiving the DNA-PK inhibitor. In some embodiments, the combination regimen comprises the steps of: (a) under the direction or control of a physician, the subject receiving the DNA-PK inhibitor prior to first receipt of the PD-L1 antibody; and (b) under the direction or control of a physician, the subject receiving the PD-L1 antibody. In some embodiments, the combination regimen comprises the steps of: (a) prescribing the subject to self-administer, and verifying that the subject has self-administered, the PD-L1 antibody prior to first administration of the DNA-PK inhibitor; and (b) administering the DNA-PK inhibitor to the subject. In some embodiments, the combination regimen comprises the steps of: (a) prescribing the subject to self-administer, and verifying that the subject has self-administered, the DNA-PK inhibitor prior to first administration of the PD-L1 antibody; and (b) administering the PD-L1 antibody to the subject. In some embodiments, the combination regimen comprises, after the subject has received the PD-L1 antibody prior to the first administration of the DNA-PK inhibitor, administering the DNA-PK inhibitor to the subject. In some embodiments, the combination regimen comprises, after the subject has received the DNA-PK inhibitor prior to first administration of the anti-PD-L1 antibody, administering the anti-PD-L1 antibody to the subject.

In a further aspect, the combination regimen comprises a lead phase, optionally followed by a maintenance phase after completion of the lead phase. As used herein, the combination treatment comprises a defined period of treatment (i.e., a first phase or lead phase). After completion of such a period or phase, another defined period of treatment may follow (i.e., a second phase or maintenance phase). In other words, upon completion of a chemotherapy treatment in patients who have stable disease or better, a strategy of maintenance could be advantageous and treat the patients until progressive disease. In certain embodiments, the maintenance can preferably include the anti-PD-L1 antibody monotherapy, more preferably avelumab monotherapy, or a combination with the DNA-PK inhibitor.

The treatment regimens differ in the lead phase and maintenance phase. In some embodiments, the anti-PD-L1 antibody and DNA-PK inhibitor are administered concurrently in either the lead or maintenance phase and optionally non-concurrently in the other phase, or the anti-PD-L1 antibody and DNA-PK inhibitor are administered non-concurrently in the lead and maintenance phase. In some embodiments, the anti-PD-L1 antibody and the DNA-PK inhibitor are administered concurrently (during the same phase) in either the lead or maintenance phase. In particular, if the anti-PD-L1 antibody and the DNA-PK inhibitor are administered concurrently in the lead phase, they are not concurrently administered in the maintenance phase again, and vice versa. In some embodiments, either the anti-PD-L1 antibody or the DNA-PK inhibitor can be additionally administered in the other phase, optionally together with chemotherapy, radiotherapy or chemoradiotherapy. In some embodiments, the anti-PD-L1 antibody and DNA-PK inhibitor are administered non-concurrently in the lead and maintenance phase, i.e., one of them is administered in the lead phase and the other one in the maintenance phase.

In some embodiments, the concurrent administration comprises the administration of the anti-PD-L1 antibody and DNA-PK inhibitor sequentially in either order or substantially simultaneously. As used herein, the concurrent administration comprises the administration of the anti-PD-L1 antibody and DNA-PK inhibitor sequentially in either order or substantially simultaneously, in each case during one and the same phase of treatment. The anti-PD-L1 antibody and DNA-PK inhibitor are administered to the patient in any order (i.e., simultaneously or sequentially) in separate compositions, formulations or unit dosage forms, or together in a single composition, formulation or unit dosage form. In certain embodiments, the anti-PD-L1 antibody is administered simultaneously in the same composition comprising the anti-PD-L1 antibody and DNA-PK inhibitor. In certain embodiments, the anti-PD-L1 antibody and DNA-PK inhibitor are administered simultaneously in separate compositions, i.e., wherein the anti-PD-L1 antibody and DNA-PK inhibitor are administered simultaneously each in a separate unit dosage form. It will be appreciated that the anti-PD-L1 antibody and DNA-PK inhibitor are administered on the same day or on different days and in any order as according to an appropriate dosing protocol. In contrast, the non-concurrent administration comprises the administration of the anti-PD-L1 antibody and DNA-PK inhibitor sequentially in two different phases of treatment, i.e., only one of them is administered in the lead phase and the other one in the maintenance phase.

The anti-PD-L1 antibody, preferably avelumab, can be given concurrently with the DNA-PK inhibitor (either alone or in combination with chemotherapy or radiotherapy or both) or sequentially, i.e., after treatment with the DNA-PK inhibitor (with or without chemotherapy or radiotherapy) has stopped (as maintenance therapy).

In some embodiments, the DNA-PK inhibitor is administered alone in the lead phase. In some embodiments, the DNA-PK inhibitor is administered concurrently with one or more therapies in the lead phase, wherein such therapies are selected from the group of an anti-PD-L1 antibody, a chemotherapy and radiotherapy. The lead phase particularly comprises the concurrent administration of the DNA-PK inhibitor and PD-L1 antibody.

In some embodiments, the anti-PD-L1 antibody is administered alone in the maintenance phase. In some embodiments, the anti-PD-L1 antibody is administered concurrently with the DNA-PK inhibitor in the maintenance phase. In some embodiments, none of them is administered in the maintenance phase. In some embodiments, there is no maintenance phase.

In some embodiments, the lead phase comprises the administration of the DNA-PK inhibitor and, after completion of the lead phase, the maintenance phase comprises the administration of the anti-PD-L1 antibody. Both, the DNA-PK inhibitor and anti-PD-L1 antibody can be administered alone, concurrently or non-concurrently with one or more chemotherapeutic agents, radiotherapy or chemoradiotherapy. The chemotherapy and/or radiotherapy are preferably administered in the lead phase.

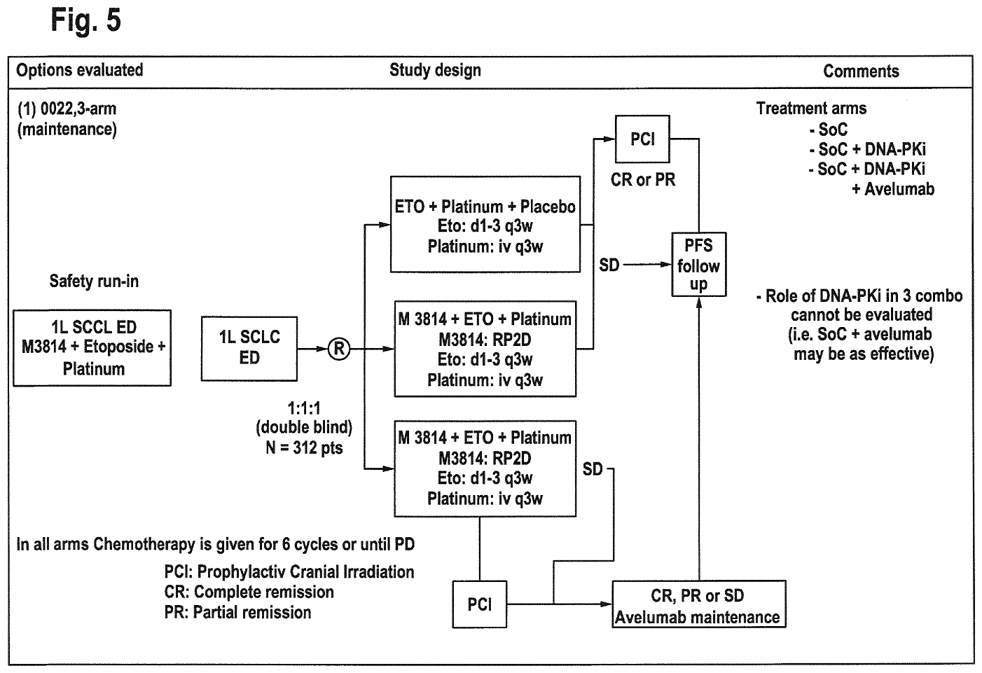

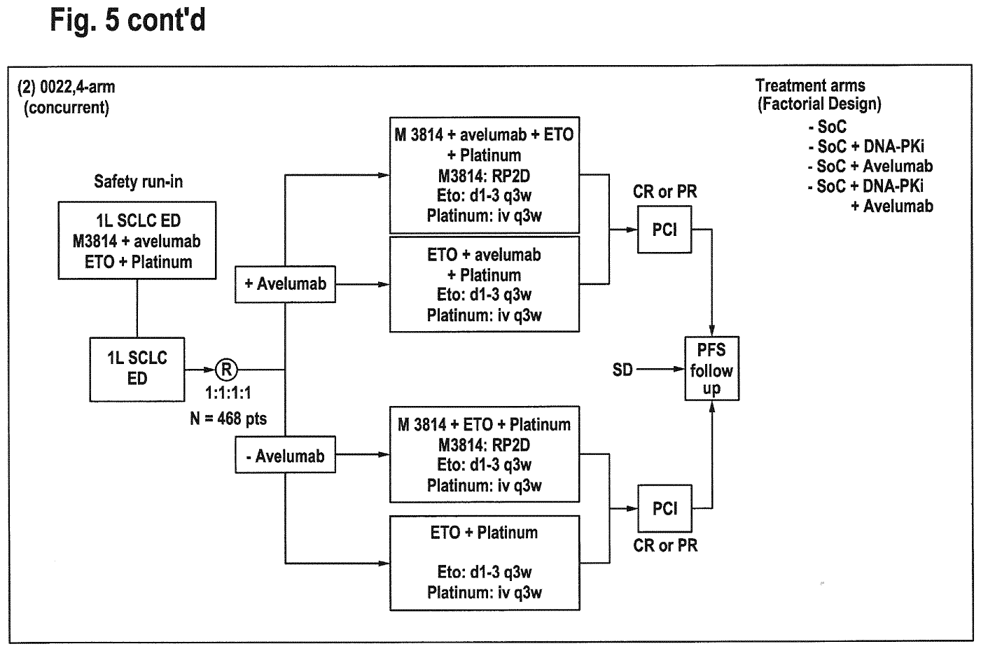

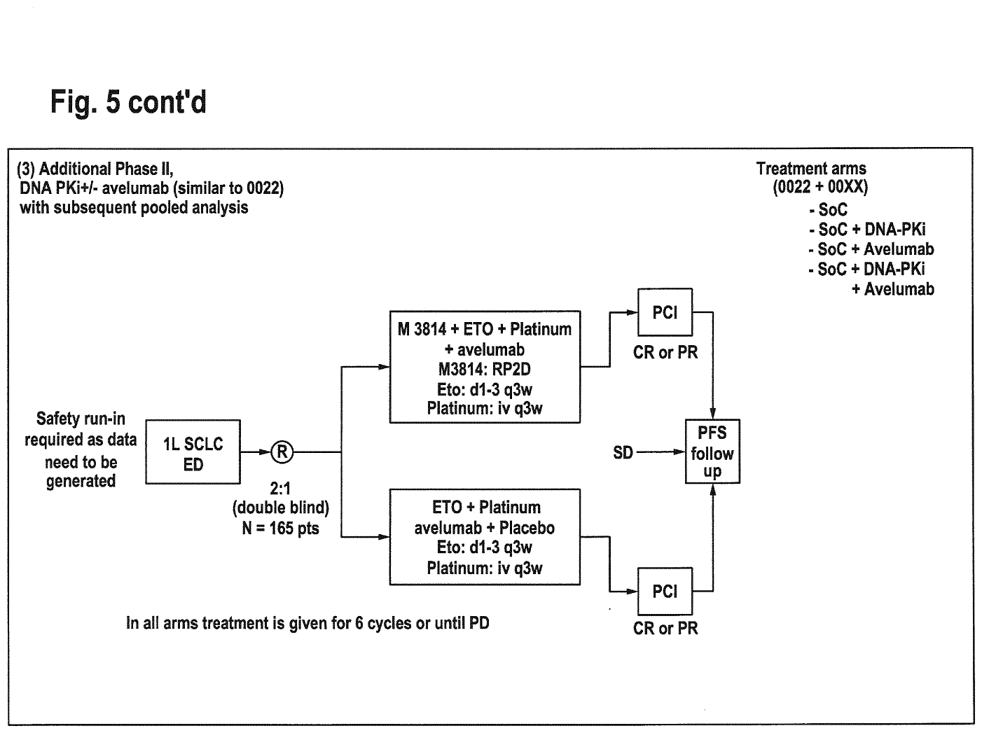

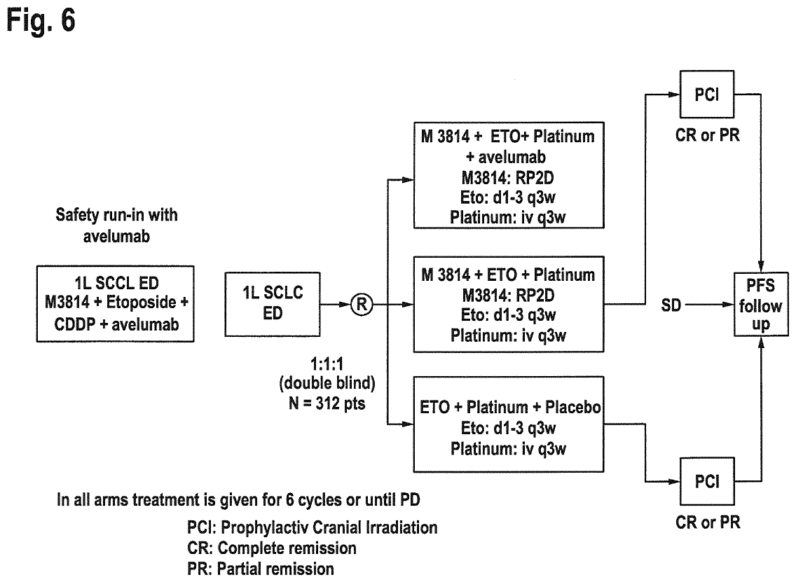

In some preferred embodiments, the present invention provides a method of treating SCLC ED in a subject during the lead and maintenance phase, wherein the lead phase comprises the concurrent administration of the DNA-PK inhibitor and etoposide, optionally together with cisplatin, and the maintenance phase comprises the administration of the anti-PD-L1 antibody, optionally together with the DNA-PK inhibitor, after completion of the lead phase. Herein, the lead phase particularly comprises the triple combination of the DNA-PK inhibitor, etoposide and cisplatin for SCLC ED treatment (see e.g., FIG. 5(1)).

In some preferred embodiments, the present invention provides a method of treating subjects with metastatic NSCLC who have progressed after the induction therapy during the second-line and consolidation treatment. Whilst the lead phase comprises the administration of the DNA-PK inhibitor in combination with the anti-PD-L1 antibody and radiotherapy, the maintenance phase comprises the administration of the anti-PD-L1 antibody, optionally together with the DNA-PK inhibitor. Herein, the lead phase particularly comprises the triple combination of the DNA-PK inhibitor, avelumab and radiotherapy.

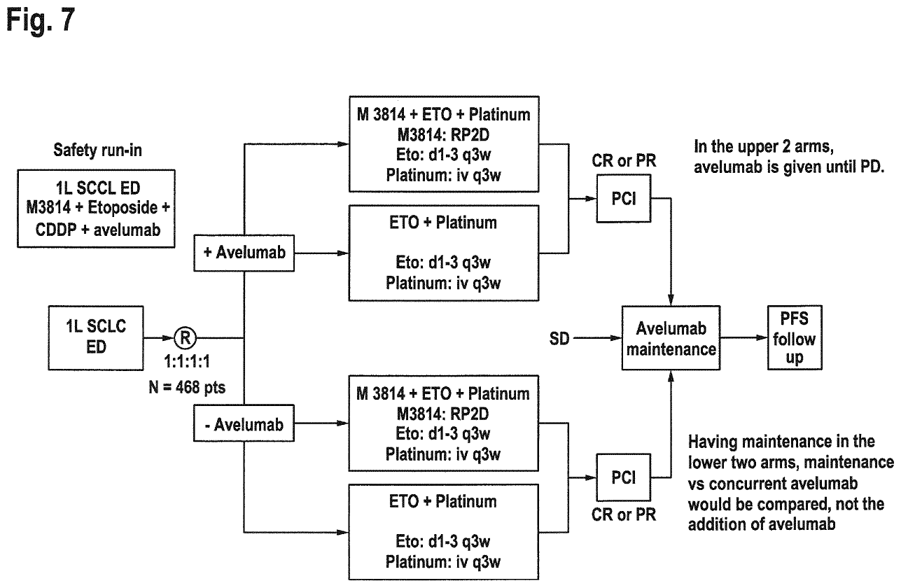

In some other preferred embodiments, the present invention provides a method of treating SCLC ED in a subject during the lead phase, wherein the lead phase comprises the concurrent administration of the anti-PD-L1 antibody, DNA-PK inhibitor and etoposide, optionally together with the cisplatin, and optionally further comprising the maintenance phase after completion of the lead phase, wherein the maintenance phase comprises the administration of the anti-PD-L1 antibody (see e.g., FIG. 5(2), 5(3) or 6). Herein, the lead phase particularly comprises the quadruple combination of the anti-PD-L1 antibody, DNA-PK inhibitor, etoposide and cisplatin for SCLC ED treatment. After completion of the lead phase, the SCLC ED treatment can be continued in the maintenance phase comprising the administration of the anti-PD-L1 antibody (see e.g., FIG. 7). The duration of treatment with the chemotherapy is in some cases capped at 6 cycles (e.g., when treating SCLC) or until progression of the malignant disease. In some embodiments, the etoposide, optionally together with the cisplatin, is administered up to 6 cycles or until progression of SCLC ED. Without being bound by any theory, after chemotherapy, residual tumor cells will continue to produce spontaneous DSBs during replication, which will make them a target for the DNA-PK inhibitor. Most patients receiving chemotherapy for SCLC will achieve at best a partial response and therefore benefit from a maintenance therapy, which combines the DNA-PK inhibitor to inhibit DSB repair occurring after chemotherapy with an immune-checkpoint inhibitor, i.e., the anti-PD-L1 antibody, to further reduce tumor burden and/or the disease recurrence.

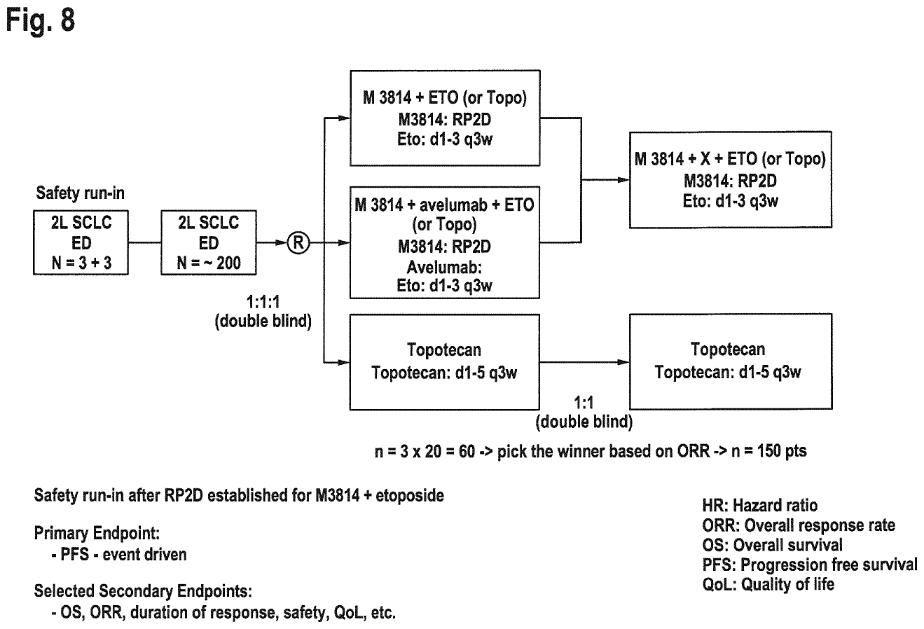

In one embodiment, SCLC is treated at second line or beyond. In particular, it includes patients with refractory SCLC (i.e., patients whose disease relapse within 3 months have an OS of ˜5.7 months, a PFS of 2.6 months and RR of ˜10%) and patients with relapsed SCLC (i.e., patients whose disease relapse after 3 months have an OS of 7.8 months and a RR ˜23%). For patients with refractory SCLC, no SoC exists, although Topotecan is widely used (see e.g., FIG. 8).

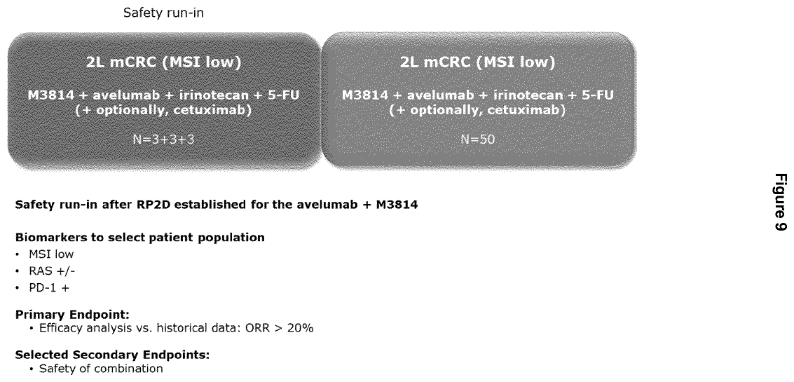

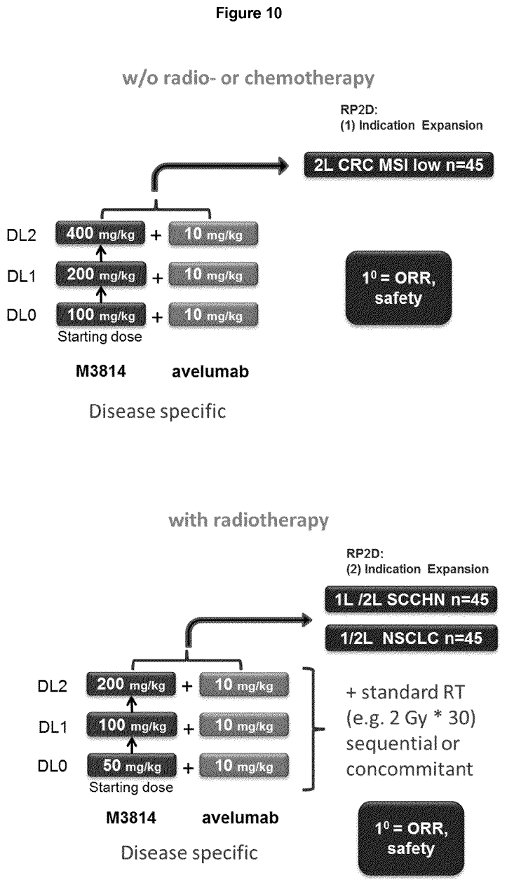

In some other preferred embodiments, the present invention provides a method of treating mCRC MSI-L during the lead phase, which comprises the concurrent administration of the anti-PD-L1 antibody, DNA-PK inhibitor, irinotecan and fluorouracil. In one embodiment, MSI low mCRC is treated second line or higher. Colorectal cancer (CRC) can be subdivided into several molecular subgroups based on, e.g., KRAS and NRAS mutational status, which has an impact on treatment (e.g., EGFR targeting vs. VEGF targeting). Another characterization is based on the microsatelite status, either stable (MSS) or instable, either low (MSI-L) or high (MSI-H). MSI-H is seen in only ˜15% of all patients with CRC but MSI-L/MSS in 85%. Earlier studies have shown that PD-x in monotherapy have no effect on MSS/MSI-L CRC patients (0% ORR) (Le et al. (2015), N Engl J Med 372: 2509) (see e.g., FIG. 9 or 10(1)).

In some other preferred embodiments, the present invention provides a method of treating NSCLC or SCCHN during the lead and maintenance phase, wherein the lead phase comprises the concurrent administration of the DNA-PK inhibitor and radiotherapy or chemoradiotherapy and, after completion of the lead phase, the maintenance phase comprises the administration of the anti-PD-L1 antibody. Herein, the lead phase particularly comprises the concurrent administration of the anti-PD-L1 antibody, DNA-PK inhibitor and radiotherapy for the NSCLC or SCCHN treatment. In one embodiment, chemoradiotherapy is followed by avelumab in the first-line treatment of NSCLC. In one embodiment, radiotherapy is administered concurrently with avelumab in the first-line treatment of NSCLC. In one preferred embodiment, chemoradiotherapy is followed by avelumab in the first-line treatment of SCCHN. In one preferred embodiment, radiotherapy is administered concurrently with avelumab in the first-line treatment of SCCHN. In one preferred embodiment, radiotherapy is administered concurrently with avelumab in the second-line treatment of recurrent SCCHN eligible for re-irradiation (40-50 Gy). Patients with recurrent/metastatic SCCHN have an OS of ˜5-7 months, a PFS of 4-5 months and RR of ˜30%. For patients with recurrent/metastatic SCCHN, no SoC exists, although metrotrexate, platins with or without fluorouracil as well as taxanes are used (see e.g., FIG. 10(2)).

Also provided herein is an anti-PD-L1 antibody for use as a medicament in combination with a DNA-PK inhibitor. Similarly provided is a DNA-PK inhibitor for use as a medicament in combination with an anti-PD-L1 antibody. Also provided is an anti-PD-L1 antibody for use in the treatment of cancer in combination with a DNA-PK inhibitor. Similarly provided is a DNA-PK inhibitor for use in the treatment of cancer in combination with an anti-PD-L1 antibody.

Also provided is a combination comprising an anti-PD-L1 antibody and a DNA-PK inhibitor. Also provided is a combination comprising an anti-PD-L1 antibody and a DNA-PK inhibitor for use as a medicament. Also provided is a combination comprising an anti-PD-L1 antibody and a DNA-PK inhibitor for the use in the treatment of cancer.

Unless explicitly stated otherwise, it shall be understood that, in the various embodiments described above, the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

Also provided is the use of a combination for the manufacture of a medicament for the treatment of cancer, comprising an anti-PD-L1 antibody and a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

The prior teaching of the present specification concerning the therapeutic combination, including the methods of using it, and all aspects and embodiments thereof, of this Section titled “Therapeutic combination and method of use thereof” is valid and applicable without restrictions to the medicament, the anti-PD-L1 antibody and/or DNA-PK inhibitor for use in the treatment of cancer as well as the combination, and aspects and embodiments thereof, of this Section, if appropriate.

Pharmaceutical Formulations and Kits

In some embodiments, the present invention provides a pharmaceutically acceptable composition comprising an anti-PD-L1 antibody. In some embodiments, the present invention provides a pharmaceutically acceptable composition comprising a DNA-PK inhibitor, preferably Compound 1, or a pharmaceutically acceptable salt thereof. In some embodiments, the present invention provides a pharmaceutically acceptable composition of a chemotherapeutic agent. In some embodiments, the present invention provides a pharmaceutical composition comprising an anti-PD-L1 antibody, a DNA-PK inhibitor and at least a pharmaceutically acceptable excipient or adjuvant. In the various embodiments described above and below, the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. In some embodiments, a composition comprising a DNA-PK inhibitor, preferably Compound 1, or a pharmaceutically acceptable salt thereof, is separate from a composition comprising an anti-PD-L1 antibody and/or a chemotherapeutic agent. In some embodiments, a DNA-PK inhibitor, preferably Compound 1, or a pharmaceutically acceptable salt thereof, and an anti-PD-L1 antibody and/or a chemotherapeutic agent are present in the same composition.

In certain embodiments, the present invention provides a composition comprising a DNA-PK inhibitor, preferably Compound 1, or a pharmaceutically acceptable salt thereof, and at least one of etoposide and cisplatin, optionally together with the anti-PD-L1 antibody. In some embodiments, a provided composition comprising a DNA-PK inhibitor, preferably Compound 1, or a pharmaceutically acceptable salt thereof, and at least one of etoposide and cisplatin is formulated for oral administration.

Exemplary such pharmaceutically acceptable compositions are described further below and herein.

Liquid dosage forms for oral administration include, but are not limited to, pharmaceutically acceptable emulsions, microemulsions, solutions, suspensions, syrups and elixirs. In addition to Compound 1, or a pharmaceutically acceptable salt thereof, and/or a chemotherapeutic agent, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, lavouring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of the anti-PD-L1 antibody, DNA-PK inhibitor, preferably Compound 1, and/or an additional chemotherapeutic agent, it is often desirable to slow absorption from subcutaneous or intramuscular injection. This may be accomplished by the use of a liquid suspension of crystalline or amorphous material with poor water solubility. The rate of absorption then depends upon its rate of dissolution that, in turn, may depend upon crystal size and crystalline form. Alternatively, delayed absorption of parenterally administered anti-PD-L1 antibody, DNA-PK inhibitor, preferably Compound 1, or a pharmaceutically acceptable salt thereof, and/or a chemotherapeutic agent, is accomplished by dissolving or suspending the compound in an oil vehicle. Injectable depot forms are made by forming microencapsule matrices of anti-PD-L1 antibody, DNA-PK inhibitor, preferably Compound 1, or a pharmaceutically acceptable salt thereof, and/or a chemotherapeutic agent, in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories, which can be prepared by mixing the compounds of this invention with suitable non-irritating excipients or carriers such as cocoa butter, polyethylene glycol or a suppository wax, which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol and silicic acid, b) binders such as, for example, carboxymethylcellulose, alginates, gelatin, polyvinylpyrrolidinone, sucrose and acacia, c) humectants such as glycerol, d) disintegrating agents such as agar-agar, calcium carbonate, potato or tapioca starch, alginic acid, certain silicates and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The anti-PD-L1 antibody, DNA-PK inhibitor, preferably Compound 1, or a pharmaceutically acceptable salt thereof, and/or a chemotherapeutic agent, can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms, the anti-PD-L1 antibody, DNA-PK inhibitor, preferably Compound 1, or a pharmaceutically acceptable salt thereof, and/or a chemotherapeutic agent, may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the anti-PD-L1 antibody, DNA-PK inhibitor, preferably Compound 1, or a pharmaceutically acceptable salt thereof, and/or a chemotherapeutic agent, include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Typically, the anti-PD-L1 antibodies or antigen-binding fragments according to the invention are incorporated into pharmaceutical compositions suitable for administration to a subject, wherein the pharmaceutical composition comprises the anti-PD-L1 antibodies or antigen-binding fragments thereof, and a pharmaceutically acceptable carrier. In many cases, it is preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the anti-PD-L1 antibodies or antigen-binding fragments thereof.

The compositions of the present invention may be in a variety of forms. These include, for example, liquid, semi-solid and solid dosage forms, such as liquid solutions (e.g., injectable and infusible solutions), dispersions or suspensions, tablets, pills, powders, liposomes, and suppositories. The preferred form depends on the intended mode of administration and therapeutic application. Typical preferred compositions are in the form of injectable or infusible solutions, such as compositions similar to those used for passive immunization of humans. The preferred mode of administration is parenteral (e.g., intravenous, subcutaneous, intraperitoneal, or intramuscular). In a preferred embodiment, the anti-PD-L1 antibody or antigen-binding fragment thereof is administered by intravenous infusion or injection. In another preferred embodiment, the anti-PD-L1 antibody or antigen-binding fragment thereof is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active anti-PD-L1 antibody or antigen-binding fragment thereof in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, the preferred methods of preparation are vacuum drying and freeze-drying that yield a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion, and by the use of surfactants. Prolonged absorption of injectable compositions can be brought about by including in the composition an agent that delays absorption, for example, monostearate salts and gelatin.

In one embodiment, avelumab is a sterile, clear, and colorless solution intended for IV administration. The contents of the avelumab vials are non-pyrogenic, and do not contain bacteriostatic preservatives. Avelumab is formulated as a 20 mg/mL solution and is supplied in single-use glass vials, stoppered with a rubber septum and sealed with an aluminum polypropylene flip-off seal. For administration purposes, avelumab must be diluted with 0.9% sodium chloride (normal saline solution). Tubing with in-line, low protein binding 0.2 micron filter made of polyether sulfone (PES) is used during administration.

In a further aspect, the invention relates to a kit comprising an anti-PD-L1 antibody and a package insert comprising instructions for using the anti-PD-L1 antibody in combination with a DNA-PK inhibitor to treat or delay progression of a cancer in a subject. Also provided is a kit comprising a DNA-PK inhibitor and a package insert comprising instructions for using the DNA-PK inhibitor in combination with an anti-PD-L1 antibody to treat or delay progression of a cancer in a subject. Also provided is a kit comprising an anti-PD-L1 antibody and a DNA-PK inhibitor, and a package insert comprising instructions for using the anti-PD-L1 antibody and a DNA-PK inhibitor to treat or delay progression of a cancer in a subject. In the various embodiments of the kit described above, the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6. The kit can comprise a first container, a second container and a package insert, wherein the first container comprises at least one dose of a medicament comprising the anti-PD-L1 antibody, the second container comprises at least one dose of a medicament comprising the DNA-PK inhibitor, and the package insert comprises instructions for treating a subject for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1 expression by an immunohistochemical (IHC) assay.

The prior teaching of the present specification concerning the therapeutic combination, including the methods of using it, and all aspects and embodiments thereof, of the previous Section titled “Therapeutic combination and method of use thereof” is valid and applicable without restrictions to the pharmaceutical formulations and kits, and aspects and embodiments thereof, of this Section titled “Pharmaceutical formulations and kits”, if appropriate.

Further Diagnostic, Predictive, Prognostic and/or Therapeutic Methods

The disclosure further provides diagnostic, predictive, prognostic and/or therapeutic methods, which are based, at least in part, on determination of the identity of the expression level of a marker of interest. In particular, the amount of human PD-L1 in a cancer patient sample can be used to predict whether the patient is likely to respond favorably to cancer therapy utilizing the therapeutic combination of the invention.

Any suitable sample can be used for the method. Non-limiting examples of such include one or more of a serum sample, plasma sample, whole blood, pancreatic juice sample, tissue sample, tumor lysate or a tumor sample, which can be an isolated from a needle biopsy, core biopsy and needle aspirate. For example, tissue, plasma or serum samples are taken from the patient before treatment and optionally on treatment with the therapeutic combination of the invention. The expression levels obtained on treatment are compared with the values obtained before starting treatment of the patient. The information obtained may be prognostic in that it can indicate whether a patient has responded favorably or unfavorably to cancer therapy.

It is to be understood that information obtained using the diagnostic assays described herein may be used alone or in combination with other information, such as, but not limited to, expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient, and the like. In a particular aspect, the expression level can be used in a diagnostic panel each of which contributes to the final diagnosis, prognosis, or treatment selected for a patient.

Any suitable method can be used to measure the PD-L1 peptide, DNA, RNA, or other suitable read-outs for PD-L1 levels, examples of which are described herein and/or are well known to the skilled artisan.

In some embodiments, determining the PD-L1 level comprises determining the PD-L1 expression. In some preferred embodiments, the PD-L1 level is determined by the PD-L1 peptide concentration in a patient sample, e.g., with PD-L1 specific ligands, such as antibodies or specific binding partners. The binding event can, e.g., be detected by competitive or non-competitive methods, including the use of a labeled ligand or PD-L1 specific moieties, e.g., antibodies, or labeled competitive moieties, including a labeled PD-L1 standard, which compete with marker proteins for the binding event. If the marker specific ligand is capable of forming a complex with PD-L1, the complex formation can indicate PD-L1 expression in the sample. In various embodiments, the biomarker protein level is determined by a method comprising quantitative western blot, multiple immunoassay formats, ELISA, immunohistochemistry, histochemistry, or use of FACS analysis of tumor lysates, immunofluorescence staining, a bead-based suspension immunoassay, Luminex technology, or a proximity ligation assay. In a preferred embodiment, the PD-L1 expression is determined by immunohistochemistry using one or more primary anti-PD-L1 antibodies.

In another embodiment, the biomarker RNA level is determined by a method comprising microarray chips, RT-PCR, qRT-PCR, multiplex qPCR or in-situ hybridization. In one embodiment of the invention, a DNA or RNA array comprises an arrangement of poly-nucleotides presented by or hybridizing to the PD-L1 gene immobilized on a solid surface. For example, to the extent of determining the PD-L1 mRNA, the mRNA of the sample can be isolated, if necessary, after adequate sample preparation steps, e.g., tissue homogenization, and hybridized with marker specific probes, in particular on a microarray platform with or without amplification, or primers for PCR-based detection methods, e.g., PCR extension labeling with probes specific for a portion of the marker mRNA.

Several approaches have been described for quantifying PD-L1 protein expression in IHC assays of tumor tissue sections (Thompson et al. (2004) PNAS 101(49): 17174; Thompson et al. (2006) Cancer Res. 66: 3381; Gadiot et al. (2012) Cancer 117: 2192; Taube et al. (2012) Sci Transl Med 4, 127ra37; and Toplian et al. (2012) New Eng. J Med. 366 (26): 2443). One approach employs a simple binary end-point of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells. The level of PD-L1 mRNA expression may be compared to the mRNA expression levels of one or more reference genes that are frequently used in quantitative RT-PCR, such as ubiquitin C. In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in non-malignant cells of the same type or in a section from a matched normal tissue.

In a preferred embodiment, the efficacy of the therapeutic combination of the invention is predicted by means of PD-L1 expression in tumor samples. Immunohistochemistry with anti-PD-L1 primary antibodies can be performed on serial cuts of formalin fixed and paraffin embedded specimens from patients treated with an anti-PD-L1 antibody, such as avelumab.

This disclosure also provides a kit for determining if the combination of the invention is suitable for therapeutic treatment of a cancer patient, comprising means for determining a protein level of PD-L1, or the expression level of its RNA, in a sample isolated from the patient and instructions for use. In another aspect, the kit further comprises avelumab for immunotherapy. In one aspect of the invention, the determination of a high PD-L1 level indicates increased PFS or OS when the patient is treated with the therapeutic combination of the invention. In one embodiment of the kit, the means for determining the PD-L1 protein level are antibodies with specific binding to PD-L1, respectively.

In still another aspect, the invention provides a method for advertising an anti-PD-L1 antibody in combination with a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6, comprising promoting, to a target audience, the use of the combination for treating a subject with a cancer based on PD-L1 expression in samples taken from the subject. Promotion may be conducted by any means available. In some embodiments, the promotion is by a package insert accompanying a commercial formulation of the therapeutic combination of the invention. The promotion may also be by a package insert accompanying a commercial formulation of the anti-PD-L1 antibody, DNA-PK inhibitor or another medicament (when treatment is a therapy with the therapeutic combination of the invention and a further medicament). Promotion may be by written or oral communication to a physician or health care provider. In some embodiments, the promotion is by a package insert where the package insert provides instructions to receive therapy with the therapeutic combination of the invention after measuring PD-L1 expression levels, and in some embodiments, in combination with another medicament. In some embodiments, the promotion is followed by the treatment of the patient with the therapeutic combination of the invention with or without another medicament. In some embodiments, the package insert indicates that the therapeutic combination of the invention is to be used to treat the patient if the patient's cancer sample is characterized by high PD-L1 biomarker levels. In some embodiments, the package insert indicates that the therapeutic combination of the invention is not to be used to treat the patient if the patient's cancer sample expresses low PD-L1 biomarker levels. In some embodiments, a high PD-L1 biomarker level means a measured PD-L1 level that correlates with a likelihood of increased PFS and/or OS when the patient is treated with the therapeutic combination of the invention, and vice versa. In some embodiments, the PFS and/or OS is decreased relative to a patient who is not treated with the therapeutic combination of the invention. In some embodiments, the promotion is by a package insert where the package inset provides instructions to receive therapy with avelumab in combination with a DNA-PK inhibitor after first measuring PD-L1 levels. In some embodiments, the promotion is followed by the treatment of the patient with avelumab in combination with a DNA-PK inhibitor with or without another medicament. Further methods of advertising and instructing, or business methods applicable in accordance with the invention are described (for other drugs and biomarkers) in US 2012/0089541, for example.

The prior teaching of the present specification concerning the therapeutic combination, including the methods of using it, and all aspects and embodiments thereof, of the previous Section titled “Therapeutic combination and method of use thereof” is valid and applicable without restrictions to the methods and kits, and aspects and embodiments thereof, of this Section titled “Further diagnostic, predictive, prognostic and/or therapeutic methods”, if appropriate.

All the references cited herein are incorporated by reference in the disclosure of the invention hereby.

It is to be understood that this invention is not limited to the particular molecules, pharmaceutical compositions, uses and methods described herein, as such matter can, of course, vary. It is also to be understood that the terminology used herein is for the purpose of describing particular embodiments only and is not intended to limit the scope of the present invention, which is only defined by the appended claims. The techniques that are essential according to the invention are described in detail in the specification. Other techniques which are not described in detail correspond to known standard methods that are well known to a person skilled in the art, or the techniques are described in more detail in cited references, patent applications or standard literature. Provided that no other hints in the application are given, they are used as examples only, they are not considered to be essential according to the invention, but they can be replaced by other suitable tools and biological materials.

Although methods and materials similar or equivalent to those described herein can be used in the practice or testing of the present invention, suitable examples are described below. Within the examples, standard reagents and buffers that are free from contaminating activities (whenever practical) are used. The examples are particularly to be construed such that they are not limited to the explicitly demonstrated combinations of features, but the exemplified features may be unrestrictedly combined again provided that the technical problem of the invention is solved. Similarly, the features of any claim can be combined with the features of one or more other claims. The present invention having been described in summary and in detail, is illustrated and not limited by the following examples.

### EXAMPLES

### Example 1: DNA-PK Inhibitor in Combination with Avelumab

The combination potential of M3814 (Compound 1) and Avelumab was elaborated in mice using the murine colon tumor model MC38. This model allows the use of immunocompetent mice, a necessary requirement to study the T-cell mediated antitumor effect of Avelumab. The experimental set up included the induction of MC38 tumors in C57BL6/N mice by injection of 1×10⁶ tumor cells into the right flank of the animals. Tumor growth was followed over time by measuring length and width using a caliper. When tumors were established to an average size of 50-100 mm³, mice were subdivided in 4 treatment groups with 10 animals each, and treatment started. This day was defined as day 0. Group 1 received vehicle treatment. Group 2 received M3814 orally once daily at 150 mg/kg in a volume of 10 ml/kg. Group 3 received avelumab intravenously once daily at 400 μg/mouse in a volume of 5 ml/kg on days 3, 6 and 9. Group 4 received M3814 orally once daily at 150 mg/kg in a volume of 10 ml/kg and avelumab intravenously once daily at 400 μg/mouse in a volume of 5 ml/kg on days 3, 6 and 9.

As a result of the study, the combined treatment of M3814 and avelumab was significantly superior to either of the monotherapy treatments (FIG. 3). A Kaplan-Meyer evaluation of the data revealed that the median time the tumors of the respective treatment groups needed to double in size as compared to their initial volume at day 0 was 6 days for Group 1, 10 days for Group 2, 13 days for Group 3, and 20 days for group 4. The respective T/C values calculated at day 13 were 47% for Group 2, 60% for Group 3, and 21% for Group 4. The treatment was overall well tolerated.

### Example 2: DNA-PK Inhibitor in Combination with Avelumab and Radiotherapy

The combination potential of M3814 (Compound 1), avelumab and radiotherapy was elaborated in mice using the murine colon tumor model MC38. This model allows the use of immunocompetent mice, a necessary requirement to study the T-cell mediated antitumor effect of avelumab. The experimental set up included the induction of MC38 tumors in C57BL6/N mice by injection of 1×10⁶ tumor cells into the right flank of the animals. Tumor growth was followed over time by measuring length and width using a caliper. When tumors were established to an average size of 50-100 mm³, mice were subdivided in 4 treatment groups with 10 animals each, and treatment started. This day was defined as day 0. Group 1 received Ionizing radiation (IR) at a daily dose of 2 Gy for 5 consecutive days and vehicle treatment. Group 2 received IR at a daily dose of 2 Gy for 5 consecutive days and M3814 orally once daily at 100 mg/kg in a volume of 10 ml/kg for 5 consecutive days, 30 min prior to each IR fraction. Group 3 received IR at a daily dose of 2 Gy for 5 consecutive days and avelumab intravenously once daily at 400 μg/mouse in a volume of 5 ml/kg on days 8, 11 and 14. Group 4 received IR at a daily dose of 2 Gy for 5 consecutive days and M3814 orally once daily at 100 mg/kg in a volume of 10 ml/kg for 5 consecutive days, 30 min prior to each IR fraction and avelumab intravenously once daily at 400 μg/mouse in a volume of 5 ml/kg on days 8, 11 and 14.

As a result of the study the combined treatment of M3814, avelumab and IR was significantly superior to M3814 and IR as well as avelumab and IR (FIG. 4). A Kaplan-Meyer evaluation of the data revealed that the median time the tumors of the respective treatment groups needed to double in size as compared to their initial volume at day 0 was 10 days for Group 1, 21 days for Group 2, 10 days for Group 3, and not reached for Group 4 by study end on day 28 because 60% of the animals did not reach the respective tumor volume. The treatment was overall well tolerated.

### Example 3: Combination Study with DNA-PK Inhibitor and Avelumab

This example illustrates a clinical trial study to evaluate safety, efficacy, pharmacokinetics and pharmacodynamics of a DNA-PK inhibitor (M3814) in combination with avelumab (MSB0010718C) in patients with previously treated MSI low/MSS stable CRC.

This study is an open-label, multi-center, dose escalation trial designed to estimate the maximum tolerated dose (MTD) and select the recommended phase 2 dose (RP2D) of DNA-PKi when given in combination with avelumab. Once the MTD of DNA-PKi administered in combination with avelumab is estimated (dose finding portion), the dose expansion phase will be opened to further characterize the combination in term of safety profile, anti-tumor activity, pharmacokinetics, pharmacodynamics and biomarker modulation. Protocol design is set forth in Table 1.

The Dose Finding Phase will estimate the MTD and RP2D in patients with CRC who have received prior systemic therapy for advanced disease, including bevacizumab, cetuximab, 5-fluorouracil, irinotecan and oxaliplatin. Dose finding will follow a classical 3+3 design with up to 5 potential dose levels (DL) to be tested, shown in Table 1.

The Dose Escalation Phase will lead to the identification of an Expansion Test Dose for DNA-PKi in combination with avelumab in patients with CRC who have received prior systemic therapy for their advanced disease. The Expansion Test Dose will be either the MTD (i.e., the highest dose of DNA-PKi when given in combination with avelumab associated with the occurrence of DLTs in <33% of patients) or the RP2D, i.e., the highest tested dose that is declared safe and tolerable by the investigators and sponsor. Once the Expansion Test Dose is identified, the Dose Expansion Phase will be opened, and DNA-PKi in combination with avelumab will be evaluated in up to approximately 20-40 patients with previously treated CRC in one disease specific cohort and previously treated patients with SCLC.

TABLE 1

| Arms | Assigned Interventions |
|---|---|
| Dose finding phase | Group 1: avelumab 10 mg/kg IV Q2W; DNA-PKi |
| | 200 mg oral BID |
| | Group 2: avelumab 10 mg/kg IV Q2W; DNA-PKi |
| | 300 mg oral BID |
| | Group 3: avelumab 10 mg/kg IV Q2W; DNA-PKi |
| | 400 mg oral BID |
| | Group 4: avelumab ? mg/kg IV Q2W; DNA-PKi ? |
| | mg oral BID* |
| | Group 5: avelumab ? mg/kg IV Q2W; DNA-PKi ? |
| | mg oral BID* |
| Dose expansion | Group 1: DNA-PKi and avelumab at RP2D given |
| phase | to patients with previously treated MSI |
| | low/MSS CRC |
| | Group 2: DNA-PKi and avelumab at RP2D given to |
| | patients with previously treated SCLC |

*Potential for intermediate doses of DNA-PKi or lower doses of avelumab to be decided by the safety monitoring committee

Inclusion Criteria: Histologically or cytologically confirmed advanced MSI low/MSS stable CRC (group 1) or SCLC (group 2). Mandatory archival formalin fixed, paraffin embedded (FFPE) tumor tissue block from primary tumor resection specimen (all patients). For Extension Cohort only, mandatory de-novo tumor biopsy from a locally recurrent or metastatic lesion unless obtained from a procedure performed within 6 months of study entry and if the patient has received no intervening systemic anticancer treatment. At least one measureable lesion as defined by RECIST version 1.1. Age ≥18 years. Eastern Cooperative Oncology Group (ECOG) performance status 0 or 1. Adequate bone marrow function, renal and liver functions. The number of patients to be enrolled in the Dose Finding Phase will depend on the observed safety profile, and the number of tested dose levels. Up to approximately 95 patients (including Dose Finding Phase and Dose Expansion Phase) are projected to be enrolled in the study.

Study Treatment: DNA-PKi will be given orally (PO) twice daily (BID) without food intake, on a continuous dosing schedule. Avelumab will be given as a 1-hour intravenous infusion (IV) every two weeks (Q2W). In all patients, treatment with study drugs may continue until confirmed disease progression, patient refusal, patient lost to follow up, unacceptable toxicity, or the study is terminated by the sponsor, whichever comes first. In order to mitigate avelumab infusion-related reactions, a premedication regimen of 25 to 50 mg IV or oral equivalent diphenhydramine and 650 mg IV or oral equivalent acetaminophen/paracetamol (as per local practice) may be administered approximately 30 to 60 minutes prior to each dose of avelumab. This may be modified based on local treatment standards and guidelines, as appropriate.

Tumor Assessment: Anti-tumor activity will be assessed by radiological tumor assessments at 6-week intervals, using RECIST version 1.1. Complete and partial responses will be confirmed on repeated imaging at least at 4 weeks after initial documentation. After 6-12 months from enrollment in the study, tumor assessments should be conducted less frequently, i.e., at 12-week intervals. In addition, radiological tumor assessments will also be conducted whenever disease progression is suspected (e.g., symptomatic deterioration), and at the time of End of Treatment/Withdrawal (if not done in the previous 6 weeks). If radiologic imaging shows PD, tumor assessment should be repeated at least ≥4 weeks later in order to confirm PD. Brain

Computerized Tomography (CT) or Magnetic Resonance Imaging (MRI) scans are required at baseline and when there is a suspected brain metastasis. Bone scan (bone scintigraphy) or 18fluorodeoxyglucose-positron emission tomography/CT (18FDG-PET/CT) are required at baseline, then every 16 weeks only if bone metastases are present at baseline. Otherwise, bone imaging is required only if new bone metastases are suspected. Bone imaging is also required at the time of confirmation of CR for patients who have bone metastases.

Pharmacokinetic/Immunogenicity Assessments: PK/immunogenicity sampling will be collected.

Exploratory Biomarker Assessments: A key objective of the biomarker analyses that will be performed in this study is to investigate biomarkers that are potentially predictive of treatment benefit with the combination of DNA-PKi and avelumab. In addition, biomarker studies of tumor and blood biospecimens will be carried out to help further understand the mechanism of action of the DNA-PKi in combination with avelumab, as well as potential mechanisms of resistance. Tumor biospecimens from archived tissue samples and metastatic lesions will be used to analyze candidate DNA, RNA, or protein markers, or a relevant signature of markers, for their ability to identify those patients who are most likely to benefit from treatment with the study drugs. Markers that may be analyzed include, but not be limited to, PD-L1 expression tumor-infiltrating CD8+ T lymphocytes and T-cell receptor gene sequence quantitation. Optional tumor biopsies obtained upon disease progression will be used to investigate acquired mechanisms of resistance. Only core needle or excisional biopsies, or resection specimen are suitable.

Peripheral Blood: Specimens will be retained as whole blood, serum and plasma in a biobank for exploratory biomarker assessments, unless prohibited by local regulation or by decision of the Institutional Review Board or Ethics Committee. Samples may be used to identify or characterize cells, DNA, RNA or protein markers known or suspected to be of relevance to the mechanisms of action, or the development of resistance to DNA-PKi and avelumab. These include biomarkers that may aid in the identification of those patients who might preferentially benefit from treatment with avelumab in combination with DNA-PKi, including but not limited to, biomarkers related to anti-tumor immune response or target modulation, such as soluble VEGF-A, IL-8, IFNγ and/or tissue FoxP3, PD-1 and PD-L2. Biospecimens should be obtained pre-dose and at the same time as PK samples whenever possible.

### Example 4: Combination Study with DNA-PKi, Avelumab and Chemotherapy

This example illustrates a clinical trial study to evaluate safety, efficacy, pharmacokinetics, and pharmacodynamics of DNA-PKi (M3814) and avelumab (MSB0010718C) in combination with etoposide (triple combination—group 1), and cisplatin and etoposide (quadruple combination group 2) in patients with SCLC. In some cases, cisplatin can be replaced by carboplatin, while cisplatin/carboplatin is referred to as platinum in this Example.

This study is an open-label, multi-center, dose escalation trial designed to estimate the maximum tolerated dose (MTD) and select the recommended phase 2 dose (RP2D) of DNA-PKi when given in combination as part of a triple combination or as part of a quadruple combination. Once the MTD and/or RP2D of DNA-PKi administered in combination with avelumab and etoposide is estimated (dose finding portion), the dose expansion phase will be opened to further characterize the combination in term of safety profile, anti-tumor activity, pharmacokinetics, pharmacodynamics and biomarker modulation. Once the dose escalation of the triple combination has been completed dose escalation of the quadruple combination will start. Protocol design is set forth in Table 2a or 2b.

The Dose Finding Phase will estimate the MTD and/or RP2D in patients with SCLC extensive disease who have received prior systemic therapy for advanced disease, including carboplatin/cisplatin in combination with etoposide or irinotecan. Dose finding will follow a classical 3+3 design with up to 5 potential dose levels (DL) to be tested, shown in Table 2a or 2b.

The Dose Escalation Phase will lead to the identification of an Expansion Test Dose for DNA-PKi in combination with avelumab and etoposide in patients with SCLC who have received prior systemic therapy for their advanced disease. The Expansion Test Dose will be either the MTD (i.e., the highest dose of DNA-PKi when given in combination with avelumab and etoposide associated with the occurrence of DLTs in <33% of patients) or the RP2D, i.e., the highest tested dose that is declared safe and tolerable by the investigators and sponsor. Once the Expansion Test Dose is identified, the Dose Expansion Phase will be opened, and DNA-PKi in combination with avelumab and etoposide will be evaluated in up to approximately 20-40 patients with previously treated SCLC. Following the completion of the triple combination dose escalation, a similar scheme will be used for the evaluation of DNA-PKi, avelumab, etoposide and cisplatin in patients with previously untreated SCLC ED.

TABLE 2a

| Arms | Assigned Interventions |
|---|---|
| Dose finding phase | Group 1: avelumab 10 mg/kg IV Q2W; DNA-PKi |
| | 100 mg oral BID |
| | Group 2: avelumab 10 mg/kg IV Q2W; DNA-PKi |
| | 200 mg oral BID |
| | Group 3: avelumab 10 mg/kg IV Q2W; DNA-PKi |
| | 300 mg oral BID |
| | Group 4: avelumab ? mg/kg IV Q2W; DNA-PKi ? |
| | mg oral BID* |
| | Group 5: avelumab ? mg/kg IV Q2W; DNA-PKi ? |
| | mg oral BID* |
| Dose expansion | Group 1: DNA-PKi and avelumab at RP2D when |
| phase | combined with etoposide given to patients with |
| | previously treated SCLC |
| | Group 2: DNA-PKi and avelumab at RP2D when |
| | combined with etoposide and platinum given to |
| | patients with previously untreated SCLC |
| | extensive disease |

*Potential for intermediate doses of DNA-PKi or lower doses of avelumab to be decided by the safety monitoring committee
Etoposide and etoposide/cisplatin will be given in standard doses as part of the standard of care.

TABLE 2b

| Arms | Assigned Interventions |
|---|---|
| Dose finding phase | Group 1: avelumab 800 mg IV Q2W; DNA-PKi |
| | 100 mg oral BID |
| | Group 2: avelumab 800 mg IV Q2W; DNA-PKi |
| | 200 mg oral BID |
| | Group 3: avelumab 800 mg IV Q2W; DNA-PKi |
| | 300 mg oral BID |
| | Group 4: avelumab 800 mg IV Q2W; DNA-PKi ? |
| | mg oral BID* |
| | Group 5: avelumab 800 mg IV Q2W; DNA-PKi ? |
| | mg oral BID* |
| Dose expansion | Group 1: DNA-PKi and avelumab at RP2D when |
| phase | combined with etoposide given to patients with |
| | previously treated SCLC |
| | Group 2: DNA-PKi and avelumab at RP2D when |
| | combined with etoposide and platinum given to |
| | patients with previously untreated SCLC |
| | extensive disease |

*Potential for intermediate doses of DNA-PKi to be decided by the safety monitoring committee
Etoposide and etoposide/cisplatin will be given in standard doses as part of the standard of care.

Inclusion Criteria: Histologically or cytologically confirmed SCLC. Mandatory archival formalin fixed, paraffin embedded (FFPE) tumor tissue block from primary tumor resection specimen (all patients). For Extension Cohort Group 1 only, mandatory de-novo tumor biopsy from a locally recurrent or metastatic lesion unless obtained from a procedure performed within 6 months of study entry and if the patient has received no intervening systemic anticancer treatment. At least one measureable lesion as defined by RECIST version 1.1. Age ≥18 years. Eastern Cooperative Oncology Group (ECOG) performance status 0 or 1. Adequate bone marrow function, renal and liver functions. The number of patients to be enrolled in the Dose Finding Phase will depend on the observed safety profile, and the number of tested dose levels. Up to approximately 95 patients (including Dose Finding Phase and Dose Expansion Phase) are projected to be enrolled in the study.

Study Treatment: DNA-PKi will be given orally (PO) twice daily (BID) without food intake, on a continuous dosing schedule. Avelumab will be given as a 1-hour intravenous infusion (IV) every two weeks (Q2W). Etoposide will be given IV or orally on days 1, 2 and 3 repeated every 3^{rd} week. Platinum will be given on day 1 every 3^{rd} week. In all patients in group 1, treatment with study drugs may continue until confirmed disease progression, patient refusal, patient lost to follow up, unacceptable toxicity, or the study is terminated by the sponsor, whichever comes first. In group 2, patients without PD will stop treatment after 6 cycles. Patients with partial or complete remission can receive thorax irradiation and or prophylactic cranial irradiation according to institutional guidelines. After 6 cycles of chemotherapy, all patients without progressive disease can be given avelumab alone or in combination with DNA-PKi as maintenance treatment until progression. In order to mitigate avelumab infusion-related reactions, a premedication regimen of 25 to 50 mg IV or oral equivalent diphenhydramine and 650 mg IV or oral equivalent acetaminophen/paracetamol (as per local practice) may be administered approximately 30 to 60 minutes prior to each dose of avelumab. This may be modified based on local treatment standards and guidelines, as appropriate.

Tumor Assessment: Anti-tumor activity will be assessed by radiological tumor assessments at 6-week intervals, using RECIST version 1.1. Complete and partial responses will be confirmed on repeated imaging at least at 4 weeks after initial documentation. After 6-12 months from enrollment in the study, tumor assessments should be conducted less frequently, i.e., at 12-week intervals. In addition, radiological tumor assessments will also be conducted whenever disease progression is suspected, and at the time of End of Treatment/Withdrawal (if not done in the previous 6 weeks). If radiologic imaging shows PD, tumor assessment should be repeated at least N1 weeks later in order to confirm PD. Brain Computerized Tomography (CT) or Magnetic Resonance Imaging (MRI) scans are required at baseline and when there is a suspected brain metastasis. Bone scan (bone scintigraphy) or 18fluorodeoxyglucose-positron emission tomography/CT (18FDG-PET/CT) are required at baseline, then every 16 weeks only if bone metastases are present at baseline. Otherwise, bone imaging is required only if new bone metastases are suspected. Bone imaging is also required at the time of confirmation of CR for patients who have bone metastases.

Pharmacokinetic/Immunogenicity Assessments: PK/immunogenicity sampling will be collected.

Exploratory Biomarker Assessments: A key objective of the biomarker analyses that will be performed in this study is to investigate biomarkers that are potentially predictive of treatment benefit with the combination of DNA-PKi and avelumab. In addition, biomarker studies of tumor and blood biospecimens will be carried out to help further understand the mechanism of action of the DNA-PKi in combination with avelumab, as well as potential mechanisms of resistance. Tumor biospecimens from archived tissue samples and metastatic lesions will be used to analyze candidate DNA, RNA, or protein markers, or a relevant signature of markers, for their ability to identify those patients who are most likely to benefit from treatment with the study drugs. Markers that may be analyzed include, but not be limited to, PD-L1 expression tumor-infiltrating CD8+ T lymphocytes and T-cell receptor gene sequence quantitation. Optional tumor biopsies obtained upon disease progression will be used to investigate acquired mechanisms of resistance. Only core needle or excisional biopsies, or resection specimen are suitable.

Peripheral Blood: Specimens will be retained as whole blood, serum and plasma in a biobank for exploratory biomarker assessments, unless prohibited by local regulation or by decision of the Institutional Review Board or Ethics Committee. Samples may be used to identify or characterize cells, DNA, RNA or protein markers known or suspected to be of relevance to the mechanisms of action, or the development of resistance to DNA-PKi and avelumab when given in combination with etoposide or etoposide/platinum. These include biomarkers that may aid in the identification of those patients who might preferentially benefit from treatment with avelumab in combination with DNA-PKi, including but not limited to, biomarkers related to anti-tumor immune response or target modulation, such as soluble VEGF-A, IL-8, IFNγ and/or tissue FoxP3, PD-1 and PD-L2. Biospecimens should be obtained pre-dose and at the same time as PK samples whenever possible.

### Example 5: Combination Study with DNA-PKi, Avelumab and Radiotherapy with or without Chemotherapy

This example illustrates a clinical trial study to evaluate safety, efficacy, pharmacokinetics and pharmacodynamics of DNA-PKi (M3814) and avelumab (MSB0010718C) in combination with radiotherapy (RT) (triple combination—group 1) and chemo-radiotherapy (CRT) (quadruple combination group 2) in patients with SCCHN or other cancers such as, for example, esophageal cancer. The chemo-backbone for CRT is often cisplatin alone but it can also be combined with other drugs such as but not limited to 5-fluoruracil.

This study is an open-label, multi-center, dose escalation trial designed to define the maximum tolerated dose (MTD) and select the recommended phase 2 dose (RP2D) of DNA-PKi when given in combination as part of a triple combination or as part of a quadruple combination. Once the MTD and/or RP2D of DNA-PKi administered in combination with avelumab and RT is estimated (dose finding portion), the dose expansion phase will be opened to further characterize the combination in term of safety profile, anti-tumor activity, pharmacokinetics, pharmacodynamics and biomarker modulation. Once the dose escalation of the triple combination has been completed, dose escalation of the quadruple combination (CRT) will start. Protocol design is set forth in Table 3a or 3b.

The Dose Finding Phase will estimate the MTD and RP2D (group 1) in patients with malignancies localized supra-diaphragmatic treated with fractionated RT given with curative intend who have not receive prior systemic therapy. Dose finding will follow a classical 3+3 design with up to 5 potential dose levels (DL) to be tested, shown in Table 3a or 3b.

The Dose Escalation Phase will lead to the identification of an Expansion Test Dose for DNA-PKi in combination with avelumab and RT in patients with SCCHN who have not received prior systemic therapy for their disease. The Expansion Test Dose will be either the MTD (i.e., the highest dose of DNA-PKI when given in combination with avelumab and RT associated with the occurrence of DLTs in <33% of patients) or the RP2D, i.e., the highest tested dose that is declared safe and tolerable by the investigators and sponsor. Once the Expansion Test Dose is identified, the Dose Expansion Phase will be opened, and DNA-PKi in combination with avelumab and RT will be evaluated in up to approximately 20-40 patients with previously untreated SCCHN. Following the completion of the RT combination dose escalation, a similar scheme will be used for the evaluation of DNA-PKi, avelumab and CRT in patients with previously untreated SCCHN.

TABLE 3a

| Arms | Assigned Interventions |
|---|---|
| Dose finding phase | Group 1: avelumab 10 mg/kg IV Q2W; DNA-PKi |
| | 100 mg oral BID |
| | Group 2: avelumab 10 mg/kg IV Q2W; DNA-PKi |
| | 200 mg oral BID |
| | Group 3: avelumab 10 mg/kg IV Q2W; DNA-PKi |
| | 300 mg oral BID |
| | Group 4: avelumab ? mg/kg IV Q2W; DNA-PKi ? |
| | mg oral BID* |
| | Group 5: avelumab ? mg/kg IV Q2W; DNA-PKi ? |
| | mg oral BID* |
| Dose expansion | Group 1: DNA-PKi and avelumab at RP2D when |
| phase | combined with RT given to patients with |
| | previously untreated SCCHN |
| | Group 2: DNA-PKi and avelumab at RP2D when |
| | combined with CRT given to patients with |
| | previously untreated SCCHN |

*Potential for intermediate doses of DNA-PKi or lower doses of avelumab to be decided by the safety monitoring committee
RT and CRT will be given in standard doses per institutional guidelines as part of the standard of care.

TABLE 3b

| Arms | Assigned Interventions |
|---|---|
| Dose finding phase | Group 1: avelumab 800 mg IV Q2W; DNA-PKi |
| | 100 mg oral QD |
| | Group 2: avelumab 800 mg IV Q2W; DNA-PKi |
| | 200 mg oral QD |
| | Group 3: avelumab 800 mg IV Q2W; DNA-PKi |
| | 300 mg oral QD |
| | Group 4: avelumab 800 mg IV Q2W; DNA-PKi ? |
| | mg oral QD* |
| | Group 5: avelumab 800 mg IV Q2W; DNA-PKi ? |
| | mg oral QD* |
| Dose expansion phase | Group 1: DNA-PKi and avelumab at RP2D when |
| | combined with RT given to patients with |
| | previously untreated SCCHN |
| | Group 2: DNA-PKi and avelumab at RP2D when |
| | combined with CRT given to patients with |
| | previously untreated SCCHN |

*Potential for intermediate doses of DNA-PKi to be decided by the safety monitoring committee
RT and CRT will be given in standard doses per institutional guidelines as part of the standard of care.

Inclusion Criteria: Histologically or cytologically confirmed supra-diaphragmatic disease in the dose escalation part and untreated SCCHN in the dose expansion part. Mandatory archival formalin fixed, paraffin embedded (FFPE) tumor tissue block from primary tumor resection specimen (all patients). At least one measureable lesion as defined by RECIST version 1.1. Age ≥18 years. Eastern Cooperative Oncology Group (ECOG) performance status 0 or 1. Adequate bone marrow function, renal and liver functions. The number of patients to be enrolled in the Dose Finding Phase will depend on the observed safety profile, and the number of tested dose levels. Up to approximately 95 patients (including Dose Finding Phase and Dose Expansion Phase) are projected to be enrolled in the study.

Study Treatment: DNA-PKi will be given orally (PO) once daily (QD) without food intake, on a continuous dosing schedule. Avelumab will be given as a 1-hour intravenous infusion (IV) every two weeks (Q2W). RT will be given in daily fractions of 2 Grey (Gy) 5 times a week for 6-7 weeks. However other fractionation schedules and dose per fractions can also be envisioned. In all cases, DNA-PKi will be given 1-2 hours before RT. In all patients, avelumab alone or in combination with DNA-PKi as maintenance can be given until progression. In order to mitigate avelumab infusion-related reactions, a premedication regimen of 25 to 50 mg IV or oral equivalent diphenhydramine and 650 mg IV or oral equivalent acetaminophen/paracetamol (as per local practice) may be administered approximately 30 to 60 minutes prior to each dose of avelumab. This may be modified based on local treatment standards and guidelines, as appropriate.

Tumor Assessment: Anti-tumor activity will be assessed by radiological tumor assessments at 6-week intervals, using RECIST version 1.1. Complete and partial responses will be confirmed on repeated imaging at least at 4 weeks after initial documentation. After 6-12 months from enrollment in the study, tumor assessments should be conducted less frequently, i.e., at 12-week intervals. In addition, radiological tumor assessments will also be conducted whenever disease progression is suspected, and at the time of End of Treatment/Withdrawal (if not done in the previous 6 weeks). If radiologic imaging shows PD, tumor assessment should be repeated at least N1 weeks later in order to confirm PD. Brain Computerized Tomography (CT) or Magnetic Resonance Imaging (MRI) scans are required at baseline and when there is a suspected brain metastasis. Bone scan (bone scintigraphy) or 18fluorodeoxyglucose-positron emission tomography/CT (18FDG-PET/CT) are required at baseline, then every 16 weeks only if bone metastases are present at baseline. Otherwise, bone imaging is required only if new bone metastases are suspected. Bone imaging is also required at the time of confirmation of CR for patients who have bone metastases.

Pharmacokinetic/Immunogenicity Assessments: PK/immunogenicity sampling will be collected.

Exploratory Biomarker Assessments: A key objective of the biomarker analyses that will be performed in this study is to investigate biomarkers that are potentially predictive of treatment benefit with the combination of DNA-PKi and avelumab. In addition, biomarker studies of tumor and blood biospecimens will be carried out to help further understand the mechanism of action of the DNA-PKi in combination with avelumab, as well as potential mechanisms of resistance. Tumor biospecimens from archived tissue samples and metastatic lesions will be used to analyze candidate DNA, RNA or protein markers, or a relevant signature of markers, for their ability to identify those patients who are most likely to benefit from treatment with the study drugs. Markers that may be analyzed include, but not be limited to, PD-L1 expression tumor-infiltrating CD8+ T lymphocytes and T-cell receptor gene sequence quantitation. Optional tumor biopsies obtained upon disease progression will be used to investigate acquired mechanisms of resistance. Only core needle or excisional biopsies, or resection specimen are suitable.

Peripheral Blood: Specimens will be retained as whole blood, serum and plasma in a biobank for exploratory biomarker assessments, unless prohibited by local regulation or by decision of the Institutional Review Board or Ethics Committee. Samples may be used to identify or characterize cells, DNA, RNA or protein markers known or suspected to be of relevance to the mechanisms of action, or the development of resistance to DNA-PKi and avelumab when given in combination with etoposide or etoposide/platinum. These include biomarkers that may aid in the identification of those patients who might preferentially benefit from treatment with avelumab in combination with DNA-PKi, including but not limited to, biomarkers related to anti-tumor immune response or target modulation, such as soluble VEGF-A, IL-8, IFNγ and/or tissue FoxP3, PD-1 and PD-L2. Biospecimens should be obtained pre-dose and at the same time as PK samples whenever possible.

### Example 6: Mechanistic Explanation

As mentioned before but without wishing to be bound by any particular theory, DNA-PK inhibitor, M3814, potently and selectively blocks one of the two major pathways for repair of DNA double strand breaks (DDSB) and synergizes with ionizing radiation (IR) and chemotherapy.

There is experimental data showing that by inhibiting DNA-PK catalytic activity in the presence of DDSBs, M3814 simultaneously suppresses DNA repair and a negative regulatory signal to ATM, leading to enhanced activation of the ATM dependent signaling, including CHK2 and p53-dependent cell cycle arrest. Combination treatment of proliferating p53 wild-type cancer cells (A549, A375, H460) with a single dose of ionizing radiation (2-5Gy) and sustained exposure to M3814 induced a complete cell cycle block. Within 4-7 days of treatment cells acquired a typical senescence phenotype with large/flat morphology and β-Gal staining. Live cell imaging and BrdU labeling in A549 cells demonstrated that this phenotype is not reversible following M3814 removal, in contrast to a fully reversible senescence-like phenotype caused by selective p53 activation by MDM2 inhibitor Nutlin-3a. Isogenic p53-null A549 cells lost the ability to fully arrest their cell cycle, confirming the role of p53 in senescence induction.

Analysis of mRNA from IR/M3814 induced senescent A549 and A375 cells by the Nanostring PanCancer Immune panel revealed activation of a large group of genes from several immune response pathways, including interferon, cytokine/chemokine, and complement. Eighteen genes were commonly upregulated >3-150 fold compared to controls. These substantial changes in gene expression were built gradually and correlated with the development of senescence phenotype. Several proteins from the induced subset were measured in the cell media (Meso Scale Discovery) and confirmed that they are secreted by senescent cells in the absence of M3814. Culture media from M3814-induced senescent cells showed increased immunomodulatory effect on human PBMC-derived immune cells via live imaging.

Without wishing to be bound by any particular theory, it is believed that the observation of the ability of M3814 to substantially strengthen the ATM/p53/CHK2-dependent cell cycle arrest in response to DDSB damage and effectively induce durable premature senescence with a strong immunomodulatory secretory phenotype provides further explanation for the benefits of the combination approach to radio-immuno-therapy of cancer according to the invention.

### Example 7: Combination Study with DNA-PKi and Avelumab with or without Radiotherapy (Palliative Dose)

This example illustrates a clinical trial study with 2 parts: Part A aims to evaluate safety, efficacy, pharmacokinetics and pharmacodynamics of DNA-PKi (M3814) and avelumab (MSB0010718C) (doublet combination), and Part B aims to evaluate safety, efficacy, pharmacokinetics and pharmacodynamics of DNA-PKi (M3814) in combination with avelumab (MSB0010718C) and radiotherapy (RT) (triplet combination).

This study is an open-label, multi-center, dose escalation trial designed to define the maximum tolerated dose (MTD) and/or the recommended phase 2 dose (RP2D) of DNA-PKi when given in combination as part of a double combination and as part of a triple combination. Once the MTD and/or RP2D of DNA-PKi administered in combination with avelumab and RT is defined, a dose expansion phase will be potentially opened to further characterize the combination in term of safety profile, anti-tumor activity, pharmacokinetics, pharmacodynamics and biomarker modulation in selected patient population (i.e., pre-treated metastatic NSCLC naïve to checkpoint inhibitors, or pretreated metastatic NSCLC refractory to checkpoint inhibitors). Protocol design is set forth in Table 4.

Part A of the Dose Finding Phase will define the MTD and/or RP2D of DNA-PKi in combination with avelumab in patients with advanced or metastatic solid tumors while Part B will define the MTD and/or RP2D of DNA-PKi in combination with avelumab and palliative RT in patients with advanced or metastatic solid tumors with primary or metastatic lesions in the lung and eligible for fractionated RT. Dose finding will follow a Bayesian design with up to 4 potential dose levels (DL) of DNA-PKi to be tested for each part.

The Dose Escalation Phase will lead to the identification of an Expansion Test Dose for DNA-PKi in combination with avelumab (Part A) and in combination with avelumab and RT (Part B). The Expansion Test Dose will be either the MTD (i.e., the highest dose of DNA-PKi when given in combination with avelumab (Part A) and with avelumab and RT (Part B) and/or the RP2D, i.e., the highest tested dose that is declared safe and tolerable by the investigators and sponsor. Once the Expansion Test Dose is identified, the Dose Expansion Phase will be potentially opened, and DNA-PKi in combination with avelumab and RT will be evaluated in up to approximately 20-40 patients with previously treated metastatic NSCLC (Group 1), and DNA-PKi in combination with avelumab will be evaluated in previously treated SCLC-ED and CRC MSI low or MSS stable in approximately 20-40 patients for each group (Group 2 and 3).

TABLE 4

| Arms | Assigned Interventions |
|---|---|
| Dose finding phase | Group 1: avelumab 800 mg IV Q2W; DNA-PKi |
| (Part A) | 100 mg oral BID |
| | Group 2: avelumab 800 mg IV Q2W; DNA-PKi |
| | 200 mg oral BID |
| | Group 3: avelumab 800 mg IV Q2W; DNA-PKi |
| | 300 mg oral BID |
| | Group 4: avelumab 800 mg IV Q2W; DNA-PKi ? |
| | mg oral BID* |
| Dose finding phase | Group 1: avelumab 800 mg IV Q2W; DNA-PKi |
| (Part B) | 100 mg oral QD |
| | Group 2: avelumab 800 mg IV Q2W; DNA-PKi |
| | 200 mg oral QD |
| | Group 3: avelumab 800 mg IV Q2W; DNA-PKi |
| | 300 mg oral QD |
| | Group 4: avelumab 800 mg IV Q2W; DNA-PKi ? |
| | mg oral QD* |
| Dose expansion phase | Group 1: DNA-PKi at RP2D when combined with |
| | avelumab and RT (Part B) in patients with |
| | previously treated metastatic NSCLC |
| | Group 2: DNA-PKi at R2PD when combined with |
| | avelumab (Part A) in patients with previously |
| | treated MSI low/MSS stable CRC |
| | Group 3: DNA-PKi at R2PD when combined with |
| | avelumab (Part A) in patients with previously |
| | treated SCLC-ED |

*Potential for intermediate doses of DNA-PKi or lower doses of avelumab to be decided by the safety monitoring committee
RT will be given in standard palliative doses: 3 Gy administered in 10 fractions.

Inclusion Criteria: Histologically or cytologically confirmed advanced metastatic NSCLC eligible for radiotherapy (group 1), MSI low/MSS stable CRC (group 2) or SCLC (group 3). Mandatory archival formalin fixed, paraffin embedded (FFPE) tumor tissue block from primary tumor resection specimen (all patients). For Extension Cohort only, mandatory de-novo tumor biopsy from a locally recurrent or metastatic lesion unless obtained from a procedure performed within 6 months of study entry and if the patient has received no intervening systemic anticancer treatment. At least one measureable lesion as defined by RECIST version 1.1. Age ≥18 years. Eastern Cooperative Oncology Group (ECOG) performance status 0 or 1. Adequate bone marrow function, renal and liver functions. The number of patients to be enrolled in the Dose Finding Phase will depend on the observed safety profile, and the number of tested dose levels. Up to approximately 95 patients (including Dose Finding Phase and Dose Expansion Phase) are projected to be enrolled in the study.

Study Treatment: DNA-PKi will be given orally (PO) once daily (QD) for group 1 and twice daily (BID), on a continuous dosing schedule. Avelumab will be given as a 1-hour intravenous infusion (IV) fixed dose of 800 mg every two weeks (Q2W). In all patients, treatment with study drugs may continue until confirmed disease progression, patient refusal, patient lost to follow up, unacceptable toxicity, or the study is terminated by the sponsor, whichever comes first. In order to mitigate avelumab infusion-related reactions, a premedication regimen of 25 to 50 mg IV or oral equivalent diphenhydramine and 650 mg IV or oral equivalent acetaminophen/paracetamol (as per local practice) may be administered approximately 30 to 60 minutes prior to each dose of avelumab. This may be modified based on local treatment standards and guidelines, as appropriate.

Tumor Assessment: Anti-tumor activity will be assessed by radiological tumor assessments at 6-week intervals, using RECIST version 1.1. Complete and partial responses will be confirmed on repeated imaging at least at 4 weeks after initial documentation. After 6-12 months from enrollment in the study, tumor assessments should be conducted less frequently, i.e., at 12-week intervals. In addition, radiological tumor assessments will also be conducted whenever disease progression is suspected (e.g., symptomatic deterioration), and at the time of End of Treatment/Withdrawal (if not done in the previous 6 weeks). If radiologic imaging shows PD, tumor assessment should be repeated at least N1 weeks later in order to confirm PD. Brain Computerized Tomography (CT) or Magnetic Resonance Imaging (MRI) scans are required at baseline and when there is a suspected brain metastasis. Bone scan (bone scintigraphy) or 18fluorodeoxyglucose-positron emission tomography/CT (18FDG-PET/CT) are required at baseline, then every 16 weeks only if bone metastases are present at baseline. Otherwise, bone imaging is required only if new bone metastases are suspected. Bone imaging is also required at the time of confirmation of CR for patients who have bone metastases.

Pharmacokinetic/Immunogenicity Assessments: PK/immunogenicity sampling will be collected.

Exploratory Biomarker Assessments: A key objective of the biomarker analyses that will be performed in this study is to investigate biomarkers that are potentially predictive of treatment benefit with the combination of DNA-PKi and avelumab. In addition, biomarker studies of tumor and blood biospecimens will be carried out to help further understand the mechanism of action of the DNA-PKi in combination with avelumab, as well as potential mechanisms of resistance.

Tumor biospecimens from archived tissue samples and metastatic lesions will be used to analyze candidate DNA, RNA, or protein markers, or a relevant signature of markers, for their ability to identify those patients who are most likely to benefit from treatment with the study drugs. Markers that may be analyzed include, but not be limited to, PD-L1 expression tumor-infiltrating CD8+ T lymphocytes and T-cell receptor gene sequence quantitation. Optional tumor biopsies obtained upon disease progression will be used to investigate acquired mechanisms of resistance. Only core needle or excisional biopsies, or resection specimen are suitable.

Peripheral Blood: Specimens will be retained as whole blood, serum and plasma in a biobank for exploratory biomarker assessments, unless prohibited by local regulation or by decision of the Institutional Review Board or Ethics Committee. Samples may be used to identify or characterize cells, DNA, RNA or protein markers known or suspected to be of relevance to the mechanisms of action, or the development of resistance to DNA-PKi and avelumab. These include biomarkers that may aid in the identification of those patients who might preferentially benefit from treatment with avelumab in combination with DNA-PKi, including but not limited to, biomarkers related to anti-tumor immune response or target modulation, such as soluble VEGF-A, IL-8, IFNγ and/or tissue FoxP3, PD-1 and PD-L2. Biospecimens should be obtained pre-dose and at the same time as PK samples whenever possible.

1. A method for treating a cancer in a subject in need thereof, comprising administering to the subject an anti-PD-L1 antibody, or an antigen-binding fragment thereof, and a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6.

2. The method according to claim 1, wherein the anti-PD-L1 antibody comprises the heavy chain having amino acid sequences of SEQ ID NOs: 7 or 8 and the light chain having amino acid sequence of SEQ ID NO: 9.

3. The method according to claim 1, wherein the anti-PD-L1 antibody is avelumab.

4. The method according to claim 1, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof.

5. (canceled)

6. The method according to claim 1, wherein the cancer is selected from the group consisting of cancer of lung, head and neck, colon, neuroendocrine system, mesenchyme, breast, ovarian, pancreatic, esophagus, endometrium, prostate, cervix, brain, bladder and histological subtypes thereof, preferably non-small-cell lung cancer (NSCLC), squamous cell carcinoma of the head and neck (SCCHN), or colorectal cancer (CRC).

7. (canceled)

8. The method according to claim 1, wherein the anti-PD-L1 antibody and DNA-PK inhibitor are administered in a first-line treatment of the cancer.

9. (canceled)

10. The method according to claim 1, wherein the subject underwent at least one round of prior cancer therapy, wherein, optionally, the cancer was resistant or became resistant to prior therapy.

11-12. (canceled)

13. The method according to claim 10, wherein the cancer is selected from the group consisting of pre-treated relapsing metastatic NSCLC, unresectable locally advanced NSCLC, SCLC unsuitable for systemic treatment, pre-treated relapsing or metastatic SCCHN, recurrent SCCHN eligible for re-irradiation, and pre-treated microsatellite status instable low (MSI-L) and microsatellite status stable (MSS) metastatic colorectal cancer (mCRC).

14-18. (canceled)

19. The method according to claim 1, further comprising administering a chemotherapy (CT), radiotherapy (RT), or chemotherapy and radiotherapy (CRT) to the subject.

20. The method according to claim 19, wherein the chemotherapy is selected from the group consisting of etoposide, doxorubicin, topotecan, irinotecan, fluorouracil, a platin, an anthracycline, and a combination thereof.

21-30. (canceled)

31. The method according to claim 19, wherein the radiotherapy comprises about 35-70 Gy/20-35 fractions.

32. (canceled)

33. The method according to claim 1, which comprises a lead phase, optionally followed by a maintenance phase after completion of the lead phase.

34. The method according to claim 33, wherein the anti-PD-L1 antibody and DNA-PK inhibitor are administered concurrently in either the lead or maintenance phase and optionally non-concurrently in the other phase, or the anti-PD-L1 antibody and DNA-PK inhibitor are administered non-concurrently in the lead and maintenance phase.

35. (canceled)

36. The method according to claim 34, wherein the lead phase comprises administration of the DNA-PK inhibitor alone or concurrently with one or more therapies selected from the group of the anti-PD-L1 antibody, chemotherapy and radiotherapy; wherein, optionally, the maintenance phase comprises administration of the anti-PD-L1 antibody alone or concurrently with the DNA-PK inhibitor, or none of the anti-PD-L1 antibody and the DNA-PK inhibitor.

37-44. (canceled)

45. The method according to claim 36, wherein the lead phase comprises the concurrent administration of the anti-PD-L1 antibody, DNA-PK inhibitor, irinotecan and fluorouracil, wherein the anti-PD-L1 antibody and DNA-PK inhibitor are administered in a second-line or higher treatment of cancer, and wherein the cancer is mCRC MSI-L.

46. The method according to claim 36, wherein the lead phase comprises the concurrent administration of the DNA-PK inhibitor and radiotherapy or chemoradiotherapy, wherein the maintenance phase comprises the administration of the anti-PD-L1 antibody after completion of the lead phase, and wherein the cancer is NSCLC or SCCHN.

47. The method according to claims 7 and 36, wherein the lead phase comprises the concurrent administration of the anti-PD-L1 antibody, DNA-PK inhibitor and radiotherapy, and wherein the cancer is NSCLC or SCCHN.

48-52. (canceled)

53. A combination comprising an anti-PD-L1 antibody, or an antigen-binding fragment thereof, and a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6; and wherein the anti-PD-L1 antibody and DNA-PK inhibitor are provided in a single or separate unit dosage forms.

54-58. (canceled)

59. A kit comprising an anti-PD-L1 antibody, or an antigen-binding fragment thereof, and a DNA-PK inhibitor, and a package insert comprising instructions for using the anti-PD-L1 antibody and DNA-PK inhibitor to treat or delay progression of a cancer in a subject, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6; wherein, optionally, the kit comprises a first container, a second container and a package insert, wherein the first container comprises at least one dose of a medicament comprising the anti-PD-L1 antibody, the second container comprises at least one dose of a medicament comprising the DNA-PK inhibitor, and the package insert comprises instructions for treating a subject for cancer using the medicaments; wherein, further optionally, the instructions state that the medicaments are intended for use in treating a subject having a cancer that tests positive for PD-L1 expression by an immunohistochemical assay.

60-61. (canceled)

62. A method for advertising an anti-PD-L1 antibody, or an antigen-binding fragment thereof, in combination with a DNA-PK inhibitor, wherein the anti-PD-L1 antibody comprises a heavy chain, which comprises three complementarity determining regions having amino acid sequences of SEQ ID NOs: 1, 2 and 3, and a light chain, which comprises three complementarity determining regions having amino acid sequences of SEQ ID NOs: 4, 5 and 6, comprising promoting, to a target audience, the use of the combination for treating a subject with a cancer based on PD-L1 expression in samples taken from the subject wherein, optionally, the PD-L1 expression is determined by immunohistochemistry using one or more primary anti-PD-L1 antibodies.

63. (canceled)

64. A method for treating a cancer in a subject in need thereof, comprising administering to the subject an anti-PD-L1 antibody, or an antigen-binding fragment thereof, and a DNA-PK inhibitor, wherein the DNA-PK inhibitor is (S)-[2-chloro-4-fluoro-5-(7-morpholin-4-yl-quinazolin-4-yl)-phenyl]-(6-methoxypyridazin-3-yl)-methanol or a pharmaceutically acceptable salt thereof.
